(19) United States
(12) Patent Application Publication
(10) Pub. No.: US 2006/0270643 A1
(43) Pub. Date: Nov. 30, 2006

(54) Hyfroxyflutamide induced pathways related to androgen receptor negative prostate cancer cells

(76) Inventors: Chawnshang Chang, Pittsford, NY (US); Yi-Fen Lee, Rochester, NY (US); Wen-Jye Lin, Thornhill Ontario (CA)

Correspondence Address: NEEDLE & ROSENBERG, P.C., SUITE 1000, 999 PEACHTREE STREET, ATLANTA, GA, 30309-3915, US

(21) Appl. No.: 10/533,037

(22) PCT Filed: Oct. 31, 2003
(86) PCT No.: PCT/US03/34636

§ 371 Date: Mar. 31, 2006

Related U.S. Application Data
(60) Provisional application No. 60/423,340, filed on Oct. 31, 2002.

(51) Int. Cl. A61K 31/56; A61K 31/366; A61K 31/27; A61K 31/16

(52) U.S. Cl. 514/171; 514/629; 514/452; 514/453; 514/484; 514/512

(57) ABSTRACT

Disclosed are compositions and methods for reducing androgen receptor dependent cancer cell proliferation.

1. This application claims benefit of U.S. patent application Ser. No. 60/423,340 filed on Oct. 31, 2002, which is incorporated by reference herein in its entirety.

### I. ACKNOWLEDGEMENTS

2. This invention was made with government support under federal grants DK 60905, DK60848 awarded by the NIH. The Government has certain rights to this invention.

### II. BACKGROUND

3. While hydroxyflutamide (HF) has been used as an antiandrogen to block androgen-stimulated prostate tumor growth, the antiandrogen withdrawal syndrome that allows antiandrogens to stimulate prostate tumor growth still occurs in many patients treated with androgen ablation therapy. This was previously explained by mutations in the androgen receptor (AR) and/or modulation from AR coregulators, so that HF becomes an AR agonist. Disclosed herein, the effect of antiandrogen withdrawal is linked to the activation of the MAP kinase pathway as well as the PI3K/Akt and PI3K/Akt/Mdm2 pathway. These results indicate that combination therapies involving antiandrogens and inhibitors of the MAP kinase, PI3K/Akt, and PI3K/Akt/MdM2 pathways will be effective. Compositions and methods for treating prostate cancer are disclosed.

### III. SUMMARY

4. Disclosed are methods and compositions related to the treatment of cancers related to androgen receptor.

### IV. BRIEF DESCRIPTION OF THE DRAWINGS

5. The accompanying drawings, which are incorporated in and constitute a part of this specification, illustrate several embodiments and together with the description illustrate the disclosed compositions and methods.

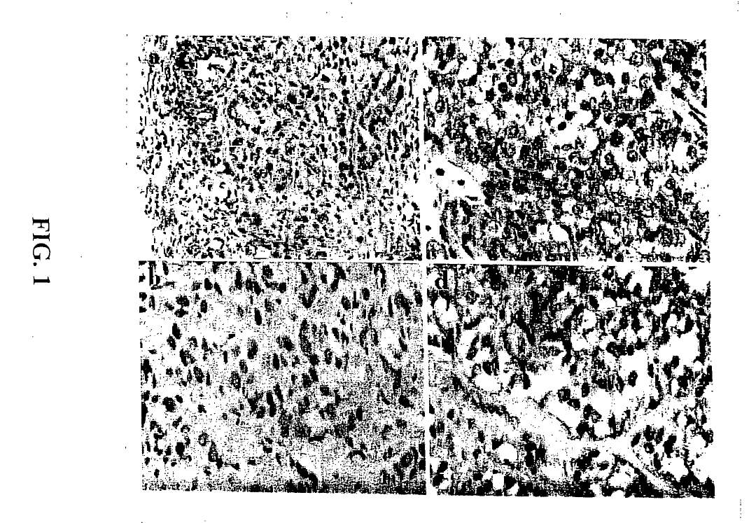

6. FIG. 1. shows elevated levels of active MAP kinase in the prostate cancer specimens from a patient whose tumors progressed on androgen ablation therapy. Prostate tumor tissue sections from the same patient with either the pre- (A and C) and post-androgen ablation plus flutamide therapy (B and D), were immunohistochemically stained for phosphorylated ERK1/2. Sections were counterstained with Mayer Hematoxylin Blue (A and B). Original magnification ×400.

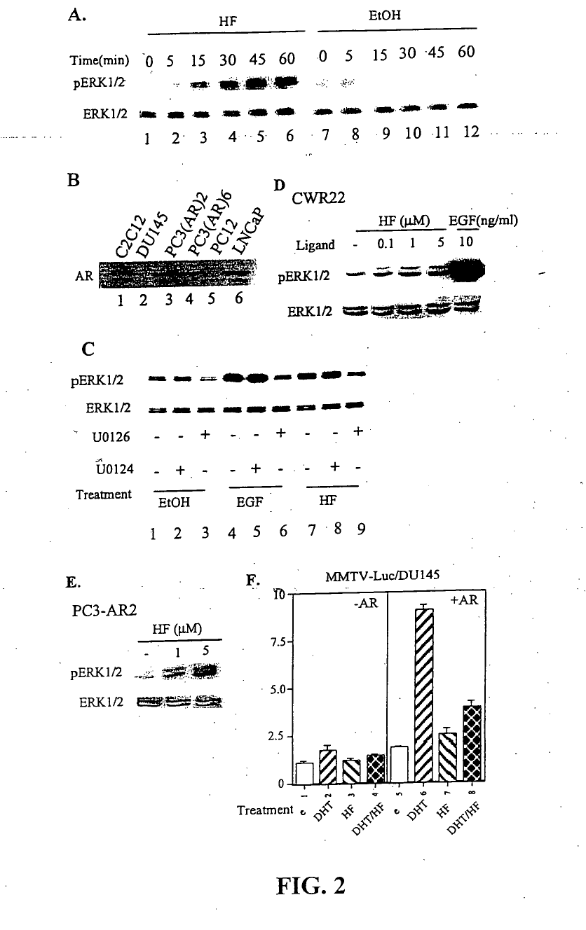

7. FIG. 2 shows activation of MAP kinase pathway by HF in prostate cancer cells. (A) DU145 cells were grown in 100 mm dishes and serum-starved for 24 hours. The cells were treated with 1 μM HF (lanes 1-6) or ethanol vehicle (lanes 7-12) for different times as indicated in the Figure. The cells were lysed on ice. Equal amounts of cell lysate were analyzed by 12% SDS-PAGE and subsequent immunoblotted with anti-phospho ERK1/2 and anti-ERK1/2 antibodies. (B) Different cell lines, at 70 to 80% confluence, were lysed and immunoblotted with anti-AR polyclonal antibody, NH27. (C) DU145 cells were pre-incubated with MEK1/2 inhibitor U0126, or U0124 before HF, EGF, and ethanol treatment. Cells were lysed and immunoblotted with anti-phospho ERK1/2 and anti-ERK1/2 antibodies. (D) CWR2 and (E) PC3-AR cells were grown in 100 mm dishes and serum-starved for 24 hours. The cells were treated with HF, EGF or ethanol vehicle for 15 mins and immunoblotted with anti-phospho-ERK1/2 and anti-ERK1/2 antibodies. (F) The antiandrogen effect of HF. MMTV-ARE-Luc reporter plasmid was cotransfected with or without pSG5AR into DU145 cells. After 18 hours, cells were treated with 1 nM DHT, 1 μM HF or both for another 18 hours, and then harvested. Cell lysates were collected and assayed for the luciferase activity.

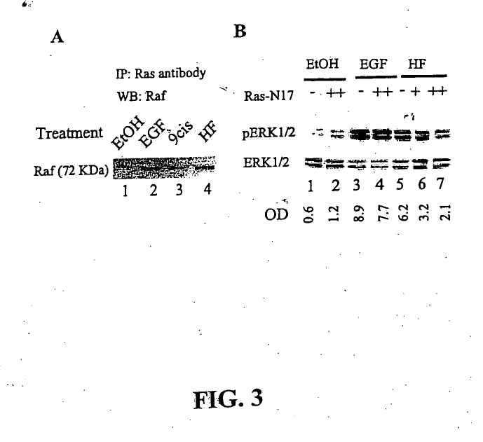

8. FIG. 3 shows HF activates Ras/Raf/MAP kinase pathway. (A) DU145 cells were treated with ethanol (lane 1), 10 ng/ml EGF (lane 2), 1 μM 9-cis retinoic acid (lane 3), and 1 μM HF (lane 4) after 24 hour serum starvation. Cells were lysed and 300 μg of total protein were immunoprecipitated with anti-Ras antibody, pulled-down by a protein A/G agarose beads, and detected with an anti-Raf antibody. (B) DU145 cells were transfected with pCDNA3.1 or pCDNA3.1-Ras N17 as indicated. After 24 hours serum starvation, the cells were treated with different ligands for 20 min, and then harvested. Western blots were performed with antibodies against phosphoERK1/2 and ERK1/2. The density of phospho-ERK1/2 shown as Optical Density (OD) were determined by the Versa Doc Imaging System (Bio-Rad) and quantified by Quantity One software.

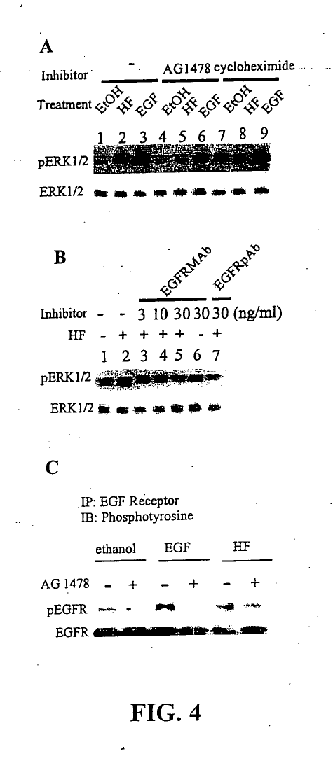

9. FIG. 4 shows inhibition of HF-mediated MAP kinase activation by EGFR inhibitors. DU145 cells were seeded and pre-incubated with (A) EGFR inhibitor tyrphostin AG1478 (100 nM) or cyclohexamide (100 μg/ml) and (B) EGFRmAb-528, or EGFRpAb-1005 for 1 hour before the EGF- or HF-treatment. After 15 min treatment, cells were lysed and immunoblotted with anti-phospho ERK1/2 and antiERK1/2 antibodies. (C) Effect of the EGF receptor (EGFR) inhibitor, tyrphostin AG1478, on HF-mediated tyrosine phosphorylation of the EGFR. Serum-Starved DU145 cells were pre-incubated with 100 μM AG1478 prior to stimulation with 10 ng/ml EGF, 1 μM HF or 10 nM of DHT for 20 min. EGFR was immunoprecipitated from the cell lysates, and their phosphotyrosine level was determined by anti-phosphotyrosine antibody. All the results were visualized using enhanced chemiluminescence.

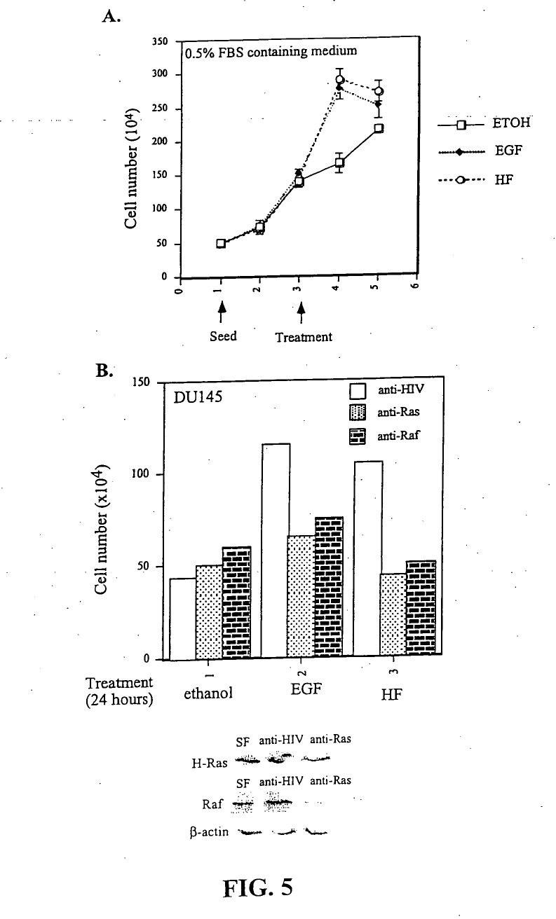

10. FIG. 5 shows HF promoted the cell proliferation. (A) DU145 cells were seeded in 10% FBS DMEM medium and 24 hours later cells were changed to serum free medium. 48 hours later changed the medium to 0.5% FBS medium and treated with ethanol, HF, or EGF. Every 24 hours, the cells were counted by hemacytometer. Cells were counted by hemacytometer every 24 hours. The results were the average from three independent experiments, and statistical analysis (t-test) was performed and showed that 24 hours of HF, as well as EGF, stimulate the DU 145 cell growth significant while compared with ethanol treatment (p<0.05). (B) DU145 cells were seeded and transfected with anti-sense oligonucleotide of Ras (IRIS 2503: 5′-TCCGTCATCGCTCCTCAGGG-3′), Raf (IIUS: 5132:5′-TCCCGCCTGTGACATGCATT-3′) and HIV (5′-TCAGTAATAGCCCCACATGG-3′) (Chen, G., Oh, S., Monia B. P., and Stacey, D. W., J. Biol. Chem., 271: 28259-28265, 1996, Monia, B. P., et al., Proc. Natl. Acad. Sci. USA, 93: 15481-15484, 1996.) by SuperFect (Qiagen). 24 hour after serum starvation, we changed the cells to medium containing 0.5% FBS, and treated the cells as described in A. Cells were counted by hemacytometer every 24 hours. The cell lysate were blotted with anti-H-Ras and Raf antibodies and β-actin was blotted for loading control.

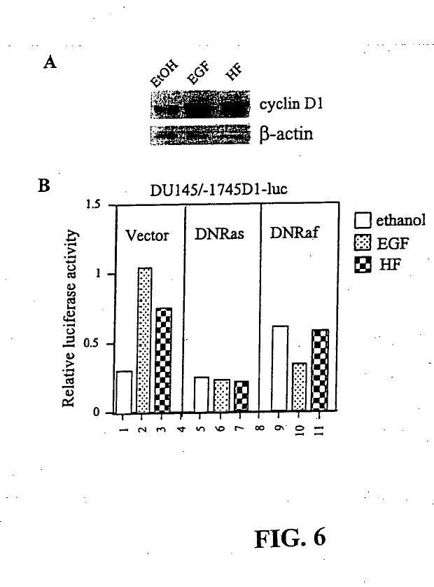

11. FIG. 6 shows HF enhanced cyclin D1 expression. (A) DU145 cells were treated with HF (1 μM), or EGF (10 ng/ml), or ethanol vehicle for 12 hours after the serum starvation. Cells were lysed and blotted with anti-cyclin D1 antibody. (B) DU145 cells were serum starved for 24 hours and then co-transfected DN Ras or DN Raf together with −1754D1 Luc reporter with SuperFect (Qiagen). After 4 hours, the medium was changed to normal medium (10% serum) for 18 hours and then changed to serum starvation condition for another 24 hours and then treated with HF, EGF, or ethanol vehicle control. Cells were lysed for luciferase activity analysis.

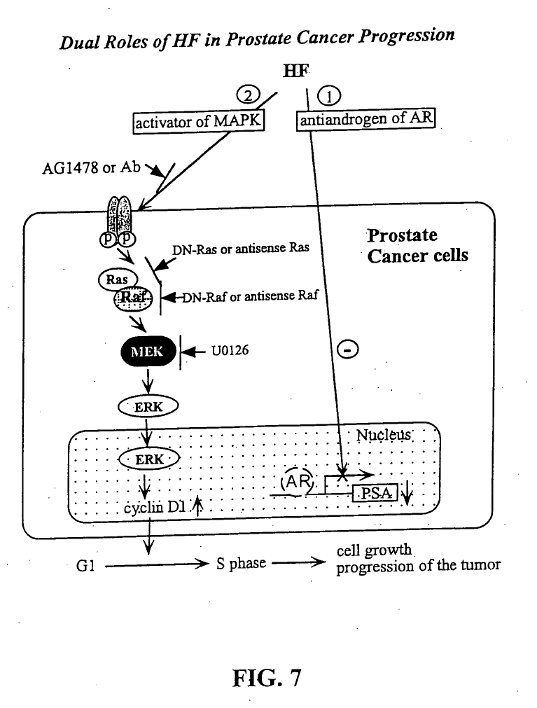

12. FIG. 7 shows a model for the HF action in prostate cancer cells.

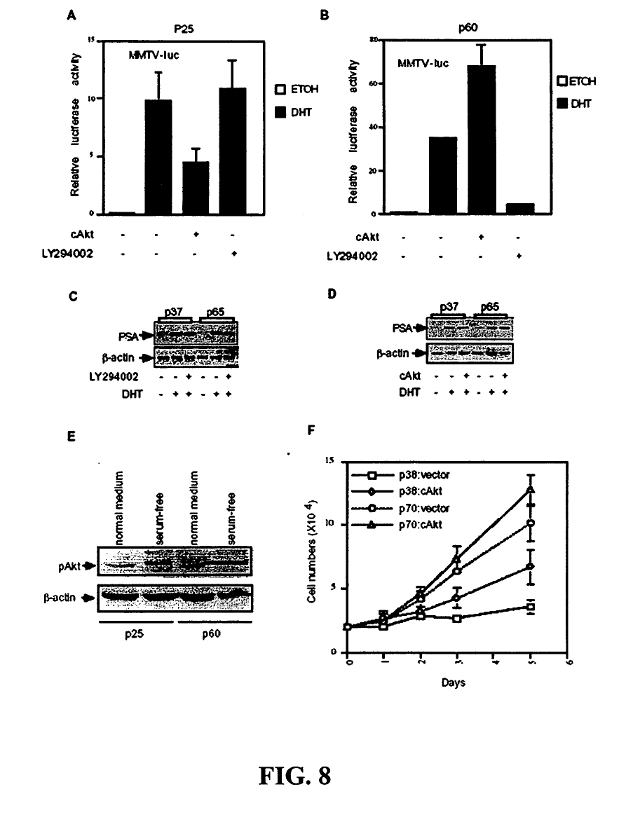

13. FIG. 8 shows the passage-dependent effect of the PI3K/Akt pathway on AR transactivation in LNCaP cells. FIG. 8A shows LNCaP cells (passage number 25 (P25)) that were transfected with MTVluc along with plasmids, as indicated, for 16 hours, and cells were then treated with ethanol (ETOH) or 10 nM DHT in the presence or absence of 20 μM LY294002 for 24 hours. The cells were harvested for luciferase assay. FIG. 8B shows the same experiment described in (A) was carried out with LNCaP cells at passage number 60 (P60). FIG. 8C shows LNCaP cells at different passage numbers were cultured in 10% CSS for 24 hours, treated with 20 μM LY294002 10 min prior to 10 nM DHT treatment for another 24 hours, and harvested for Western blot assay. FIG. 8D shows LNCaP cells at different passage numbers were transfected with vector or cAkt for 24 hours, and cells were treated with ETOH or 10 nM DHT for another 24 hours, followed by harvesting cells for Western blot assay. FIG. 8E shows that different passage numbers of LNCaP cells were cultured in the 10% FCS medium or serum-free medium for 2 days and the cells were harvested for Western blot analysis. Akt activity is determined by the levels of Akt phosphorylation (pAkt) using anti-phospho-Akt (S473) antibody. FIG. 8F shows LNCaP cells at different passages were transfected with vector or cAkt and cultured in CSS media. Cells were stained by trypan blue at different days, and cell numbers were determined as described in Experimental Procedures.

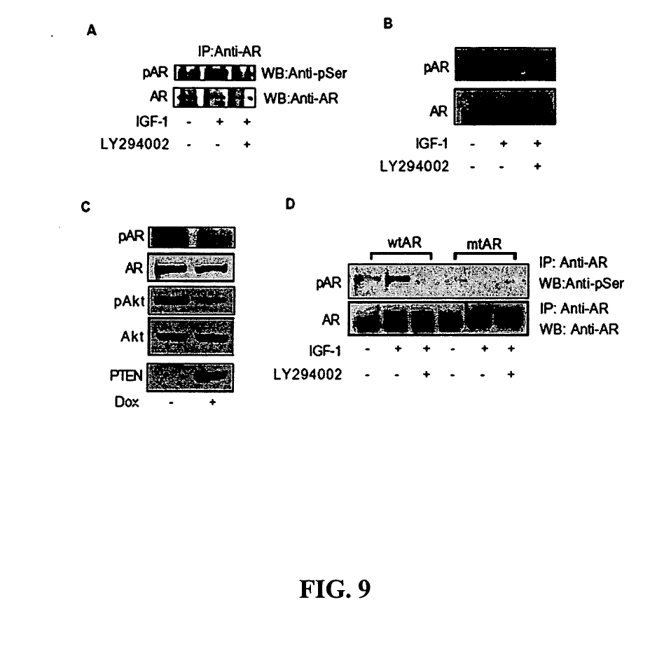

14. FIG. 9 shows that the activation of the PI3K/Akt pathway induces AR phosphorylation in vivo. FIG. 9A shows LNCaP cells at passage number 38 were serum-starved for 2 days, incubated with 20 μM LY294002 for 30 min prior to treatment with 100 μg/ml IGF-1 for 4 hours, and then harvested for immunoprecipitation with AR antibody. Anti-pSer, anti-phosphoserine antibody. FIG. 9B shows LNCaP cells at passage number 38 were treated as in (A) and harvested for Western blot analysis. Total AR protein was blotted using an anti-AR antibody (AR), and AR phosphorylation was detected using an anti-phospho-AR (S210) antibody (pAR). FIG. 9C shows PTEN-inducible LNCaP cells at passage number 40 were cultured in 10% FCS, treated with 4 μg/ml Dox for 24 hours, treated with 100 ∝g/ml IGF-1 for 4 hours, and then harvested for Western blot analysis. FIG. 9D shows COS-1 cells were transfected with wtAR or mtAR (S210A/S790A) for 16 hours, serum-starved for 24 hours, and then incubated with 20 μM LY294002 for 30 min prior to treatment with 100 μg/ml IGF-1 for 4 hours. The cells were then harvested for immunoprecipitation with anti-AR antibody and Western blot analysis. NH27, anti-AR antibody, anti-pSer, anti-phosphoserine antibody.

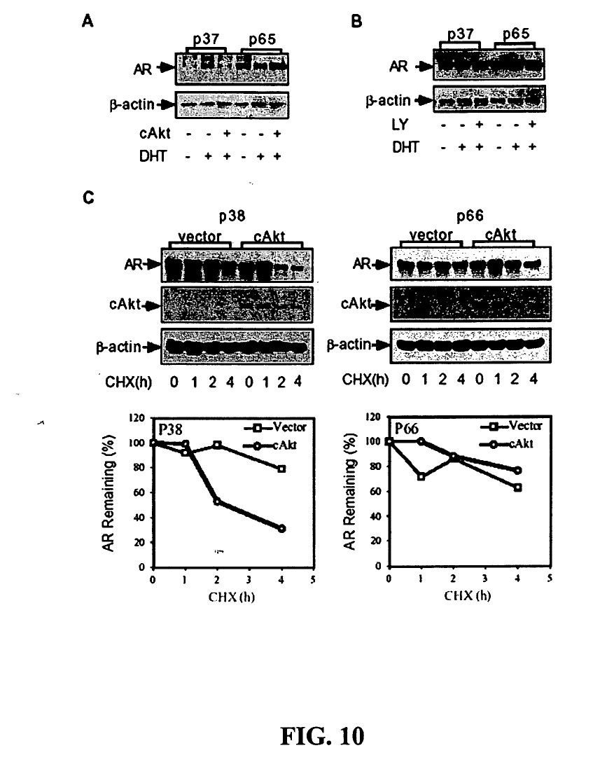

15. FIG. 10 shows distinct regulation of AR protein degradation by the PI3K/Akt pathway at various passage numbers of LNCaP cells. FIG. 10A shows LNCaP cells at different passage numbers were transfected with vector or cAkt for 24 hours, and cells were treated with ETOH or 10 nM DHT for another 24 hours, followed by harvesting for Western blot assay. FIG. 10B shows LNCaP cells at 22 different passage numbers were cultured in 10% CSS media for 24 hours, treated with 20 μM LY294002 10 min prior to 10 nM DHT treatment for another 24 hours, and harvested for Western blot assay. FIG. 10C shows LNCAP cells at different passage numbers were transfected with vector or cAkt for 24 hours, and cells were treated with 20 μg/ml cyclohexamnide (CHX) for different times, as indicated, in the 10% FCS medium, followed by harvesting for Western blot assay.

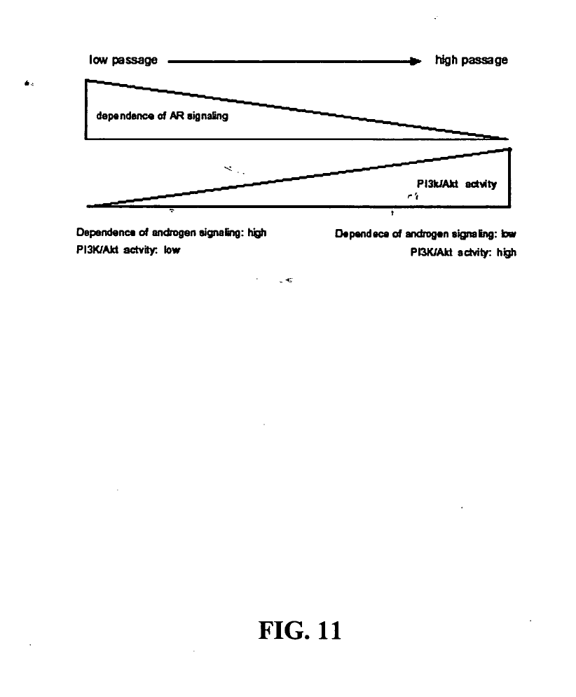

16. FIG. 11 shows the model for the PI3K/Akt pathway on AR signaling in prostate LNCaP cells. In low passage LNCaP cells, the basal activity of PI3K/Akt signaling is low, and cells are strongly dependent on androgen signaling for growth and survival. In contrast, in high passage LNCAP cells, the basal activity of the PI3K/Akt pathway is high and cells are less dependent on androgen signaling. The PI3K/Akt pathway not only provides the growth and survival signals for prostate cancer cells, but also enhances AR activity in high passage LNCaP cells.

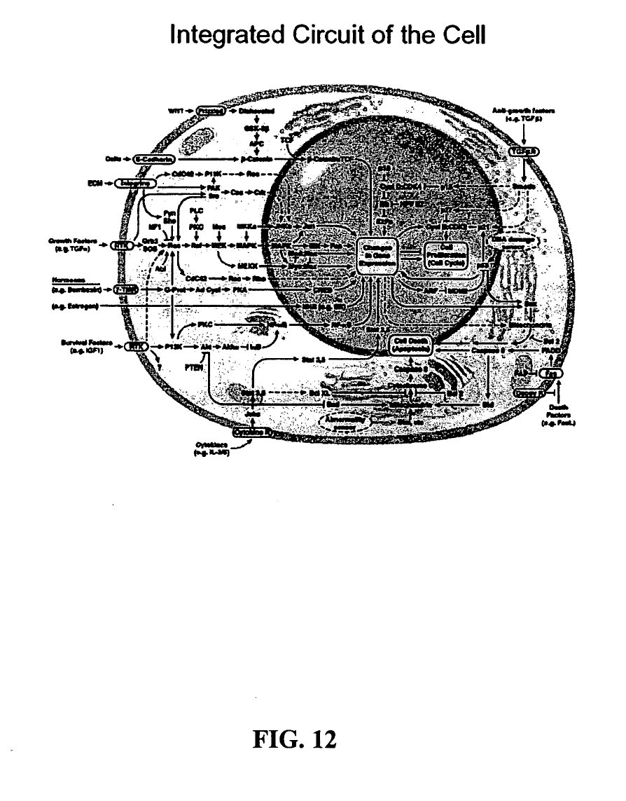

17. FIG. 12 discloses a summary of many cell signaling pathways, published in Hanahan D, Weinberg R A., “The hallmarks of cancer,” Cell. 2000 Jan. 7;100(1):57-70. This schematic sets forth many different pathways, a number of which are disclosed herein as being linked to prostate cancer through, for example, the refractory, and withdrawal mechanisms disclosed herein. As disclosed herein, modulators such as inhibitors, of the various pathways disclosed herein to be linked to prostate cancer, can be administered in combination therapies with anti-prostate cancer compounds, such as anti-androgens.

### V. DETAILED DESCRIPTION

18. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

19. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

20. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

21. In this specification and in the claims which follow, reference will be made to a number of terms which shall be defined to have the following meanings:

22. “Optional” or “optionally” means that the subsequently described event or circumstance may or may not occur, and that the description includes instances where said event or circumstance occurs and instances where it does not.

23. “Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

24. “Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

25. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular MEK inhibitor is disclosed and discussed and a number of modifications that can be made to a number of molecules including the MEK inhibitor are discussed, specifically contemplated is each and every combination and permutation of MEK inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is-understood that each of these additional steps can be performed with any specific embodiment or combination of embodiments of the disclosed methods.

26. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions and Methods

27. While hydroxyflutamide (HF) has been used as an antiandrogen to block androgen-stimulated prostate tumor growth, the antiandrogen withdrawal syndrome that allows antiandrogens to stimulate prostate tumor growth still occurs in many patients treated with androgen ablation therapy. This was previously explained by mutations in the androgen receptor (AR) and/or modulation from AR coregulators, so that HF becomes an AR agonist. Four prostate cancer patients undergoing androgen ablation therapy with flutamide were analyzed and compared for their phosph-ERK1/2 levels in prostate cancer biopsies before receiving HF and after experiencing disease progression while taking HF using immunohistochemical analysis. It was found that there was a significant increase of activated MAP kinase in prostate tumors from patients receiving HF during androgen ablation therapy. In vitro studies showed that HF induced a rapid activation of the Ras/MAP kinase pathway in human prostate cancer DU145 cells that lack the AR as well as in PC-3AR2 and CWR22 cells that express AR. Cycloheximide failed to inhibit this activation, but both AG1478, an inhibitor of the EGF receptor (EGF-R), and an EGF-R neutralizing antibody blocked this HF-mediated activation of MAP kinase, suggesting the activation of Ras/MAP kinase by HF is a membrane-initiated, non-AR mediated, and non-genomic action. The consequence of this activation is consistent with increasing cell proliferation and cyclin D1 expression. It raises a concern for using HF in the complete androgen ablation therapy in prostate cancer treatment and provides a possible pathway that might contribute to the HF withdrawal syndrome.

28. To overcome the problems associated with androgen ablation treatment and more specifically antiandrogen withdrawal syndrome, disclosed herein are compositions comprising combination therapies for the treatment of prostate cancer based on the links in prostate cancer and the pathways disclosed herein. Such treatments can be more effective than each individual treatment having a synergistic effect upon the other treatment or by overcoming a known disadvantage. Combination therapies can also involve unrelated treatments which if used together can be more effective than either alone simply by providing for the treatment of populations that would be untreated by either treatment alone. For example, the use of irradiation combined with a chemotherapeutic for the treatment of a cancer is a well-known combination therapy.

29. Specifically disclosed herein are compositions comprising combination therapies for the treatment of prostate cancer. More specifically, disclosed are treatments comprising administering to a patient an antiandrogen compound and a kinase pathway inhibitor. For example, it was found that a significant increase of activated MAP kinase in prostate tumors from patients receiving HF during androgen ablation therapy occurred. Activation of MAP kinase and its upstream regulator Ras, is linked to cell proliferation and tumor progression. Thus disclosed are compositions comprising an inhibitor of the MAP kinase or MEK pathway signal transduction pathway and an antiandrogen, such as flutamide or hydroxyflutamide.

30. The failure of antiandrogen therapy can be associated with the elevation of multiple polypeptide growth factors (Culig, Z., et al., Prostate, 28: 392-405, 1996., Culig, Z., et al., Cancer Res., 54: 5474-5478, 1994.). For example, epidermal growth factor (EGF), transforming growth factor alpha (TGFα), insulin-like growth factor-1 (IGF-I), interleukin 6, keratinocyte growth factor (KGF,) and fibroblast growth factor (FGF) family members are suggested to play important roles in fueling androgen-independent growth. Thus specifically disclosed are compositions for a combination therapy comprising an antiandrogen and a kinase pathway inhibitor, wherein the kinase pathway inhibitor is an inhibitor of a growth factor. For example the disclosed compositions can comprise Flutamide and an IGF-1 inhibitor.

31. Besides the androgen signaling that plays an essential role in survival of prostate cancer, the phosphatidylinositol 3-kinase (PI3K)/Akt pathway represents another important survival signal for prostate cancer cells. These two pathways can compensate for each other in growth regulation of prostate cancer LNCaP cells, because androgen treatment can rescue cells from apoptosis induced by application of PI3K inhibitors (Carson, J. P., et al. (1999) Cancer Res 59, 1449-1453). Thus the PI3K/Akt pathway can have distinct mechanisms to modulate AR functions in various stages of prostate cancer cells and a combined therapy of antiandrogens and anti-PI3K/Akt inhibitors can be a therapeutic approach to battle prostate cancer. Therefore, specifically disclosed are compositions comprising an antiandrogen-and-an anti-PI3K/Aktt inhibitor.

### 1. Prostate Cancer, Anti-androgens, MAP Kinase, and Akt Pathways

32. Prostate cancer is the most common noncutaneous cancer in men and the second leading cause of cancer-related death (Amanatullah, D. F., et al., Frontiers in Bioscience, 5: 372-390, 2000.). The lack of effective therapies for advanced prostate cancer reflects, in part, the lack of knowledge about the molecular mechanism involved in the development and progression of this disease (Small, E. J., Curr. Opin. Oncol., 11: 226-235,1999., Nupponen, N. and Visakorpi, T., Eur. Urol., 35: 351-354,1999.). In particular, little is known about the mechanisms that trigger the conversion of an initially androgen-dependent cancer to androgen independence. When prostate cancers first occur, they are dependent on androgens for growth and can be treated successfully with androgen ablation therapy. However, after prolonged antiandrogen therapy, eventually the cancer acquires the ability to proliferate (Brandstrom, A., Cancer Res., 54: 3594-3601, 1994., McConkey, D. J., et al., Cancer Res., 56: 5594-5599, 1996).

33. The failure of antiandrogen therapy may be associated with the elevation of multiple polypeptide growth factors (Culig, Z., et al., Prostate, 28: 392-405, 1996., Culig, Z., et al., Cancer Res., 54: 5474-5478, 1994.). For example, epidermal growth factor (EGF), transforming growth factor alpha (TGFα), insulin-like growth factor-1 (IGF-I), interleukin 6, keratinocyte growth factor (KGF,) and fibroblast growth factor (FGF) family members are suggested to play important roles in fueling androgen-independent growth. Many of these growth factors and their receptors activate Ras family members to mediate a signal transduction cascade of successive phosphorylation steps leading to the activation of mitogen-activated protein (MAP) kinases (Schlessinger, J. and Ullrich, A., Neuron, 9: 383-391, 1992.). Several studies have linked the increased activation of MAP kinases to progression of carcinomas of the kidney, liver, and prostate (Ito, Y., et al., Hepatology, 27: 951-958, 1998, Magi-Galluzzi, C., et al., Lab. Invest., 76: 37-51, 1997.). For example, Gioeli et al., found that the level of activated MAP kinase increased with increasing Gleason score and prostate tumor stage. Additionally, tumor samples from two patients that showed no activation of MAP kinase before androgen ablation therapy, developed high levels of activated MAP kinase when tumors recurred following androgen ablation (Gioeli, D., et al., Cancer Res., 59: 279-284, 1999).

34. The MAP kinase family includes the extracelluar signal-regulated kinases (ERKs, or p42/p44), the c-jun N-terminal kinases/stress-activated protein kinases (JNK/SAPKs), and p38 HOG. Reports show that the ERK and JNK pathways are stimulated by receptor protein tyrosine kinases in various cell types, however, p38 HOG is not commonly activated by growth factors (Elion E. A., Science, 281: 1625, 1998, Lewis, T. S., et al., Adv. Cancer Res., 74:-49-139, 1998, Keyse, S. M., Semin. Cell Dev. Biol., 9:143-152,1998, Thomson, S., et al., Semin. Cell Dev. Biol., 10: 205-214, 1999). Activation of MAP kinase and its upstream regulator Ras, is linked to cell proliferation and tumor progression and Voller et al., demonstrated that the finctional activation of Ras-dependent signaling could convert androgen-dependent cells to androgen independence (Voeller, H. J., et al., Mol. Endocrinol., 5: 209-216, 1991). Because of its competitive inhibition of androgen binding to the AR, hydroxyflutamide (HF) is used as an antiandrogen to treat prostate cancer. However, in vitro studies also suggested that HF could activate the mutated AR that is at times found in prostate tumors (Voeller, H. J., et al., Mol. Endocrinol., 5: 209-216, 1991, Culig, Z., et al., Mol. Endocrinol., 17: 1541-1550, 1993, Taplin, M. E., et al., N. Engl. J. Med., 332:1393-1398, 1995, and Taplin, M. E., et al., Cancer Res., 59: 2511-2515, 1999). This could explain the “flutamide withdrawal syndrome,” in which patients who experience an increase in prostatic specific antigen (PSA) while taking flutamide, have a PSA decrease after cessation of flutamide treatment (Kelly, W. K. and Scher, H. I., J Urol., 149: 607-609, 1993, Kelly, W. K., et al., Urol. Clin. North. Am., 24: 421-431, 1997, Scher, H. I. Et al., Clin. Oncol., 11: 1566-1572, 1993). However, the transient and incomplete nature of the response to antiandrogen withdrawal, as well as its failure to occur in many patients, implies there are mechanisms other than AR mutations that contribution to tumor progression.

35. Disclosed herein clinically relevant concentrations of hydroxyflutamide, 1 μM HF, rapidly activates the Ras-MAP kinase signal pathway that consequently leads to cell proliferation in an AR-independent manner. This finding not only helps explain the flutamide withdrawal syndrome but may also guide new strategies to prevent emergence of androgen independence. Disclosed are compositions, such as pharmaceutical compositions, that can be used as therapeutics in the treatment of prostate cancer. These compositions can comprise an inhibitor of the MAP kinase or MEK pathway signal transduction pathway and an antiandrogen, such as flutamide or hydroxyflutamide. These composition are based on the finding disclosed herein that refractory prostate tumor growth associated with androgens is associated with the activation of the MAP kinase pathway by the antiandrogen, such as flutamide. In addition, as discussed herein, the effects of HF are also linked herein to the (PI3K)/Akt pathway, and compositions and methods based on this finding as well are disclosed.

36. The normal prostate and prostate cancers at early stages require androgen for growth and survival. Besides the androgen signaling that plays an essential role in survival of prostate cancer, the phosphatidylinositol 3-kinase (PI3K)/Akt pathway represents another important survival signal for prostate cancer cells. These two pathways can compensate for each other in growth regulation of prostate cancer LNCaP cells, because androgen treatment can rescue cells from apoptosis induced by application of PI3K inhibitors (Carson, J. P., et al. (1999) Cancer Res 59, 1449-1453). Furthermore, activation of the PI3K/Akt pathway protects cells from apoptosis induced by serum starvation and androgen deprivation (Franke, T. F., et al. (1997) Cell 88, 435-437).

37. Herein, it is disclosed that the PI3K/Akt pathway regulates AR activity in a cell passage number-dependent manner. Specifically, PI3K/Akt pathway can suppress AR activity in androgen-dependent LNCaP cells with low passage numbers. In contrast, it can also enhance AR activity in LNCaP cells with high passage numbers. Furthermore, it is also disclosed that insulin-like growth factor-1 (IGF-1) can activate the PI3K/Akt pathway that results in the phospfrorylation of AR at S210 and S790. The consequence of these events can then change the stability of AR protein. Together, the results demonstrate that the PI3K/Akt pathway can have distinct mechanisms to modulate AR finctions in various stages of prostate cancer cells and a combined therapy of antiandrogens and anti-PI3K/Akt inhibitors can be a therapeutic approach to battle prostate cancer.

### C. Compositions

38. Disclosed are compositions comprising MAP kinase pathway inhibitors and AR inhibitors. Pharmaceutical compositions comprising MAP kinase pathway inhibitors and an antiandrogen are also disclosed. For example, disclosed are compositions comprising MAP kinase inhibitors and/or MEK kinase inhibitors, and an antiandrogen, such as hydroxyflutamide.

39. Disclosed herein MAP kinase pathway inhibitors, such as MAP kinase inhibitors or MEK inhibitors can suppress prostate cancer cell growth. Anti-proliferative therapies can be enhanced by providing reagents that target different pathways or mechanisms for cellular survival or phenotype. Thus, combinations of MAP kinase pathway inhibitors, such as MAP kinase inhibitors or MEK inhibitors and antiandrogens with other reagents for the treatment or prevention of prostate cancer are disclosed.

40. Disclosed are compositions comprising PI3K/Akt kinase pathway and Mdm2 pathway inhibitors and AR inhibitors. Pharmaceutical compositions comprising PI3K/Akt kinase pathway and Mdm2 pathway inhibitors and an antiandrogen are also disclosed. For example, disclosed are compositions comprising PI3K/Akt kinase pathway and Mdm2 pathway inhibitors, and an antiandrogen, such as hydroxyflutamide.

41. Disclosed herein PI3K/Akt kinase pathway and Mdm2 pathway inhibitors can suppress prostate cancer cell growth. Anti-proliferative therapies can be enhanced by providing reagents that target different pathways or mechanisms for cellular survival or phenotype. Thus, combinations of PI3K/Akt kinase pathway and Mdm2 pathway inhibitors and antiandrogens with other reagents for the treatment or prevention of prostate cancer are disclosed.

42. Antiandrogens typically are compositions that inhibit the activity of androgen receptor and include for example hydroxyflutamide (HF). Preferred are antiandrogens that function as HF. Also preferred are antiandrogens that function as HF and which are structurally related to HF.

43. Also disclosed are combinations of an anti-androgen, an anti-MAP or MEK kinase pathway inhibitor, and an anti PI3K/Akt or Mdm2 pathway inhibitor, or multiple inhibitors of each in any combination.

44. All of these pathways are disclosed in FIG. 12, and the various connections and effects of these pathways and between these pathways can be inferred from FIG. 12. Thus, in one aspect, inhibitors of the MAPK pathway, MEKK kinase pathway, MEK kinase pathway, Akt pathway, Mdm2 pathway, Ras pathway, and PI3K pathway are any molecules that inhibit the any of the members of these pathways shown in FIG. 12. It is clear from FIG. 12 how various molecules are connected and inhibition of one molecule can lead to inhibition of a down stream molecule for example. The disclosed composition involving combinations of various anti-prostate cancer compounds and, for example, Akt or MAP kinase pathway inhibitors, is based on the recognition of how the disclosed pathways are involved in the propagation of prostate cancer, and in particular how they are related to the refractory stage and sometimes subsequent withdrawal syndromes of prostate cancer.

### 1. Androgen Receptor

45. AR is a phosphoprotein, and the consensus phosphorylation sites found in AR indicated that AR could be a substrate for the DNA-dependent protein kinase, protein kinase A (PKA), protein kinase C (PKC), mitogen-activated kinase (MAPK), and casein kinase II (Blok et al. (1996) Endocr Res 22, 197-219). This hypothesis was supported by the observation that PKA and PKC could enhance AR transactivation (Ikonen et al. (1994) Endocrinology 135, 1359-66; Nazareth et al. (1996) J Biol Chem 271, 19900-7). Furthermore, a report also demonstrated that the HER2/Neu-MAPK pathway could phosphorylate AR that might result in much easier recruitment of AR coregulators to AR. The consequence of this signal cascade may then enhance AR transactivation (Yeh et al. (1999) Proc Natl Acad Sci USA 96, 5458-63).

46. In addition to stimulating cell growth, androgen/AR plays important roles in the promotion of cell apoptosis. For example, androgen can induce the thymic atrophy by acceleration of thymocyte apoptosis (Olsen et al. (1998) Endocrinology 139, 748-52). Androgen also causes the biphasic growth (stimulation of cell growth at 10-12-10-10M and suppression of cell growth at 10-8M) in the prostate cancer LNCaP cells, which expresses functional AR (Zhao et al. (1999) Endocrinology 140, 1205-12). AR also plays indispensible roles in the mitogen-activated protein kinase kinase kinase-1(MAPKKK1)-induced apoptosis in the prostate cancer cells (Abreu-Martin et al. (1999) Mol Cell Biol 19, 5143-54). Androgen also induces cell growth inhibition and apoptosis in the PC-3(AR)2 with stably transfected AR (Heisler et al. (1997) Mol Cell Endocrinol 126, 59-73). Finally, the tumor suppressor BRCA-1 increases the AR transactivation and promotes the androgen-induced cell death (Park et al. (2000) Cancer Res. 60, 5946-9; Yeh et al. (2000) Proc Natl Acad Sci USA 97, 11256-61). Taken together, it is well documented that androgen/AR may play dual roles in the promotion of cell growth and apoptosis.

47. The androgen receptor (AR), a member of the steroid receptor superfamily, functions as an androgen-dependent transcriptional factor (Chang et al. (1988) Science 240, 324-326). After binding to ligand, the activated AR is able to recognize palindromic DNA sequences, called androgen response elements (AREs), and form a complex with AR associated proteins to induce the expression of AR target genes. Several AR coregulators (ARAs), such as ARA24, ARA54, ARA55, ARA70, ARA160, ARA267, Rb, BRCA1 and TIFIIH, have been isolated and characterize (Hsiao et al. (1999) J. Biol. Chem. 274, 22373-22379; Kang et al. (1999) J. Biol. Chem. 274, 8570-8576; Fujimoto et al. (1999) J. Biol. Chem. 274, 8316-8321; Yeh et al. (1996) Proc. Natl. Acad. Sci. USA 93, 5517-5521; Hsiao et al. (1999) J. Biol. Chem. 274, 20229-20234; Yeh et al. (1998) Biochem. Biophys. Res. Commun. 248, 361-367; Yeh et al. (2000) Proc. Natl. Acad. Sci. USA 97, 11256-11261; Lee et al. (2000) J. Biol. Chem. 275, 9308-9313). Results from these studies suggest that coregulators not only can enhance AR transactivation, but may also be able to increase the agonist activity of antiandrogens (Miyamoto et al. (1998) Proc. Natl. Acad. Sci. USA 95, 7379-7384) and 17-β estradiol (Yeh et al. (1998) Proc. Natl. Acad. Sci. USA 95, 5527-5532.) in prostate cancer DU145 cells.

48. Sequence analysis of AR reveals two Akt consensus sequences (RXRXXS/T) (Alessi et al. (1996) FEBS Lett 399, 333-8) located at its amino-terninal domain and carboxyl-terminal domain (Ser210 (RAREAS) and Ser790 (RMRHLS)).

49. Herein it is disclosed that Akt phosphorylates AR at Ser210, inhibits AR transactivation, and blocks AR-induced apoptosis.

2. Phosphophatidylinositol 3(OH)-kinase (PI(3)K)

50. Phosphophatidylinositol 3(OH)-kinase (PI(3)K) contains the p85 regulatory domain and p110 catalytic domain. The p85 regulatory domain possesses two src-homology 2 (SH2) domains and a src-homology 3 (SH3) domain. The major role of the SH2 domain is to facilitate tyrosine kinase-dependent regulation of PI(3)K activity by increasing the catalytic activity of p110 and by inducing the recruitment of PI(3)K to the signaling complex (Carpenter et al. (1996) Biochim Biophys Acta 1288, M11-6). PI(3)K phosphorylates the inositol ring of PI(4,5)P2 at the D-3 position to form PI(3,4,5)P3. This lipid product of PI(3)K then activates Akt/Protein kinase B (PKB) in the membrane.

### 3. Akt/PKB

51. Akt/PKB, an oncoprotein, is a serine (Ser)-threonine (Thr) protein kinase. The amino terminus of Akt/PKB contains a pleckstrin homology domain, which can bind to the lipid products of PI(3)K (Franke et al. (1997) Cell 88, 435-7). Phosphorylation of Akt/PKB at Thr308 and Ser473 results in full activation of Akt/PKB kinase activity (Chan, et al. (1999) Annu Rev Biochem 68, 965-1014). The PI(3)K/Akt pathway in diverse cell types provides the survival signal that involves several pro-apoptotic proteins such as Bad (Datta et al. (1997) Cell 91, 231-41; del Peso et al. (1997) Science 278, 687-9) and Caspase-9 (Cardone et al. (1998) Science 282, 1318-21).

52. Data indicate that the role of Akt/PKB is to function as a general mediator of cell survival. Franke, T. F. et al., Cell 88,435-437 (1997). Several growth factors, such as insulin-like growth factor 1 and neurotrophins, may promote cell survival by activating the PI3K and its downstream target Akt/PKB. Akt/PKB may then phosphorylate and inhibit pro-apoptotic components, such as BAD, Caspase-9 and FKHRLI. Dana, S. R. et al., Cell 91, 231-241 (1997); Cardone, M. H. et al., Science 282, 1318-1321 (1998); Brunet, A. et al., Cell 96, 857-868 (1999). Disclosed herein, Akt/PKB phosphorylates AR.

53. Disclosed herein, Akt phosphorylates the androgen receptor (AR) at Ser210 and Ser790. A mutation at AR Ser210 results in the reversal of Akt-mediated suppression of AR transactivation. Activation of the phosphatidylinositol-3-OH kinase/Akt pathway results in the suppression of AR target genes, such as p21, and the decrease of androgen/AR-mediated apoptosis, through the inhibition of interaction between AR and AR coregulators. Disclosed is the molecular basis for cross-talk between two signaling pathways at the level of Akt and AR-AR coregulators.

### 4. Antiandrogens

54. There are a number of different types of prostate cancer therapies. For example, hormonal secretion from the hypothalamus can be modulated by LH-RH agonists, such as Lupron (Formula 3, Cas Nr 0053714-56-0)
5′oxo-Pro-His-Trp-Ser-Tyr-Dleu-Leu-Arg-Pro-NH—CH₂—CH₃ and Zoladex, (Formula 4, Cas Nr. 0065807-02-5) 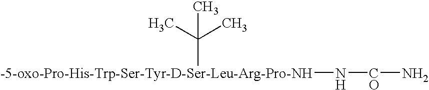
which inhibit the production of Testosterone (T) by the testes and adrenal glands. There are also anti-androgen therapeutics, such as Flutamide (Formula 5, 0013311-84-7) 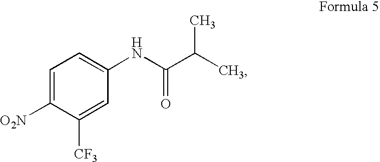
Casodex (Formula 6, Cas Nr. 0090357-06-5) 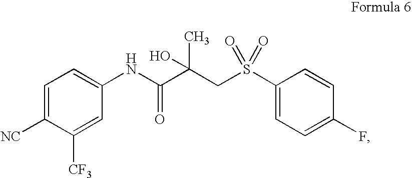
and Nilutamide (Formula 7, Cas Nr. 0063612-50-0) 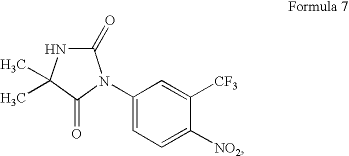
which can block the androgen binding to AR. Other therapies include the administration of 5-α reductase inhibitors, such as Proscar (Finasteride) (Formula 8 as Nr. 0098319-26-7)

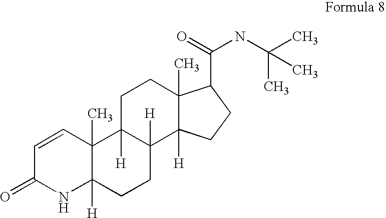

55. which can inhibit the con version of T to DHT. DHT is the mo st effective ligand for AR with higher binding affinity that T. However, this compound is generally applied for BPH patients than for prostate cancer patients.

56. Estrogen, such as DES, estradiol, and Stilphosterol Honvan, have also been used in the treatment of prostate cancer. These molecules can decrease the amount of hormones from the hypothalamus. These molecules can decrease the T synthesis from testis by inducing a negative feed-back regulation in leutinizing hormone (LH) secretion from the pituitary gland and gonadotropin releasing hormone (GnRH). secretion from the hypothalamus. Other therapeutics include Ketoconazole (Nizoral), which can inhibit the cytochrome p459 enzyme system to reduce T synthesis, and steriods such as Hydrocortisone, Aminoglutethemide (Cytadren), dexmethasome (Decadron), and Cyproterone (Androcur). Ketoconazole is usually used as a second line hormone therapy in patients with stage IV recurrent prostatic cancer. Aminoglutethimide (Cytadren) blocks adrenal steroidogenesis by inhibiting the enzymatic conversion of cholesterol to pregnenolone. Cypoterone is a steroidal antiandrogen with weak progestational activity that results in the partial suppression of pituitary gonadotropin and a decrease in serum T. The main purpose of using Hydrocortisone and Decadron is to relieve the symptoms and increase the quality of life of prostate cancer patients. It is understood that combinations of these therapeutics are performed and herein disclosed.

57. Thus, disclosed are anti-prostate cancer compounds, such as, flutamide/HF, casodex, niflutamide, finasteride, 1,25-dihydroxyl, vitamin D3, and natural products including quercetin, resveratrol, silymarin, isoflavonoids, epigallocatechin gallate (EGCG), docosahexaenoic acid (DHA) and eicosapentaenoic acid (EPA). These and others, can all be added in combination with MAP kinase pathway inhibitors or PI3K/Akt inhibitors, collectively or individually in any combination.

58. Typically, the anti-prostate cancer compounds can be provided at concentrations of less than or equal to 20 uM, 15 uM, 10, uM, 5 uM, 2 uM, 1 uM, 0.1 uM, or 0.01 uM. Typically the anti-androgens can also be provided at concentrations of less than or equal to 20 uM, 15 uM, 10, uM, 5 uM, 2 uM, 1 uM, 0.1 uM, or 0.01 uM. Typically the MAP kinase pathway inhibitors, can be administered at concentrations of less than or equal to 100 uM, 90 uM, 80 uM, 70 uM, 60 uM, 50 uM, 40 uM, 30 uM, 20 uM, 15 uM, 10, uM, 5 uM, 2 uM, 1 uM, 0.1 uM, or 0.01 uM. However those of skill in the art understand how to assay for the optimal concentration for administration in vivo, of any of the disclosed compositions, by for example, relying on disclosed cell and animal models for action, as well as by testing the compositions in vivo at various concentrations.

### 5. MAP Kinase and PI3K/Akt Pathway Inhibitors

59. The disclosed compositions and methods involve signaling through the MAP kinase pathway. The MAP kinase pathway refers to the signaling pathway which utilizes a MAP kinase for signal transduction. An inhibitor of the pathway is any molecule capable of reducing the signal transduction as compared to a control of the MAP kinase pathway. MAP kinase pathways can involve many different signaling events utilizing many different types of signaling molecules. For example, tyrosine specific protein kinase receptors can be involved. Tyrosine specific protein kinase receptors for this pathway can be involved in the growth and differentiation of cells and can typically be categorized into the following six structural subfamilies: EGF receptors, insulin-receptor/IGF- 1 receptors, NGF receptors, PDGF receptor/M-CSF receptors, FGF receptors, and VEGF receptors. The structural differences in these subfamilies and their significance can be determined by sequence and functional comparison. Typically the receptor tyrosine kinases have the common feature of an intracellular kinase domain which can be interrupted by a kinase insert region.

60. Typically upon binding to a receptor tyrosine kinase, receptor dimers (tetramers in the case of IGF-1 receptor and the insulin receptor) form which can activate the cytoplasmic catalytic domain (intracellular kinase domain) through the cross-phosphorylation of the tyrosine residues. The dimerization (tetramerization in the case of IGF-1 receptor and the insulin receptor) and subsequent phosphorylation of the tyrosine residues is referred to as autophosphorylation. The newly phosphorylated tyrosine residues serve as high affinity binding sites for intracellular signaling proteins (e.g., GTPase activating proteins (GAP), phospholipasse C-y, and Src-like nonreceptor protein tyrosine kinases), which can subsequently become phosphorylated and activated.

61. Ras proteins belong to the Ras superfamily of monomeric GTPases. Ras proteins are involved in the relay of signals from receptor kinases to the nucleus to stimulate cell proliferation and differentiation. Cellular proliferation and differentiation has been shown to be inhibited through the microinjection of neutralizing Ras antibodies.

62. Activation of Ras proteins leads to the activation of a Serine/Threonine Phosphorylation cascade (e.g., mitogen-activated protein (MAP) kinase family). The MAP kinase family (which include the extracellular-signal-regulated kinases [ERKs]) cascade involves the activation of the kinases in the family through the phosphorylation of threonine and tyrosine residues. The activated Ras protein initiates the cascade by causing the activation of the protein kinase Raf which in turn activates MAP-kinase-kinase-kinase, which in turn activates MAP-kinase-kinase (MEK), which activates MAP kinase (also known as ERK). Once MAP kinase is activated, downstream regulatory proteins can be phosphorylated causing their activation and leading to cell proliferation and differentiation.

63. MAP kinase inhibitors can be found in for example, U.S. Pat. No. 6,444,696 for Pyrazole derivatives P38 MAP kinase inhibitors, U.S. Pat. No. 6,376,527 for Pyrazole derivatives-p38 map kinase inhibitors, U.S. Pat. No. 6,316,466 for Pyrazole derivatives P-38 MAP kinase inhibitors, U.S. Pat. No. 6,316,464 for P38 MAP kinase inhibitors, U.S. Pat. No. 6,248,532 for Creba isoforms, U.S. Pat. No. 6,242,196 for Methods and pharmaceutical compositions for inhibiting tumor cell growth, U.S. Pat. No. 6,147,107 for Specific inhibition of the P42/44 mitogen activated protein (map) kinase cascade sensitizes tumor cells, U.S. Pat. No. 6,162,613 for Methods for designing inhibitors of serine/threonine-kinases and tyrosine kinases, U.S. Pat. No. 6,037,136 for Interactions between RaF proto-oncogenes and CDC25 phosphatases, and uses related thereto, U.S. Pat. No. 5,753,446 for Mitogen ERK kinase kinase (MEKK) assay U.S. Pat. No. 6,147,107 for Specific inhibition of the P42/44 mitogen activated protein (map) kinase cascade sensitizes tumor cells, U.S. Pat. No. 6,316,465 for Ophthalmic uses of PPARgamma agonists and PPARgamma antagonists, U.S. Pat. No. 6,242,196 for Methods and pharmaceutical compositions for inhibiting tumor cell growth, and U.S. Pat. No. 6,037,136 for Interactions between RaF proto-oncogenes and CDC25 phosphatases, and uses related thereto, all of which are herein incorporated by reference at least for material related to MAP kinase inhibitors.

64. An EGF receptor tyrosine kinase inhibitor can be any composition that reduces the signal transduction properties of the epidermal growth factor receptor by any means as compared to a control. For example, the reduction in activity can occur by reducing the receptor's tyrosine kinase activity or blocking the EGF receptor or blocking the phosphorylation of the EGF receptor. Such inhibitors can include but are not limited to compositions that block the extracellular domain of epidermal growth factor (EGF) thus preventing the binding of the ligand to the receptor. The monoclonal antibody C225 (Goldstein et al., (1995) Clin. Cancer Res. 1:1311-1318.) is a well known inhibitor of the EGF receptor as are the monoclonal antibody EGFRmAb-528 and the polyclonal antibody EGFRpAb-1005. Additional antibody inhibitors include but are not limited to the monoclonal antibodies LA22, LA58, and LA90 which were derived from the deposited hybridomas ATCC HB10342, ATCC HB 10343, and ATCC HB 10344 respectively (U.S. Pat. No. 5,459,061).

65. A Tyrosine Kinase inhibitor can be any composition that reduces tyrosine kinase activity as compared to a control. For example, phosphorylation activity can be reduced by being competitive inhibiting ATP binding or via an allosteric interaction with the enzyme (Levitzki et al., (1995) Science 267: 1782-1788.). Similarly, the small molecule tyrosine kinase inhibitors Tyrphostin AG1478 and Cp-358-774 prevent the phosphorylation of the tyrosine residues on EGF receptor (Moyer et al., (1997) Cancer Res. 57:4838-4848.). Also disclosed are trkA, trkB, and trK inhibitors, and it is understood that embodiments that do not include these are also disclosed.

66. Representative tyrosine kinase inhibitors can be found in for example, U.S. Pat. No. 6,455,534 for Bicyclic compounds capable of inhibiting tyrosine kinases of the epidermal growthfactor receptor family, U.S. Pat. No. 6,448,277 for VEGF receptor tyrosine kinase inhibitors, U.S. Pat. Nos. 6,420,382, 6,306,874, and 6,313,138 for Tyrosine kinase inhibitors, U.S. Pat. No. 6,333,322 for Nitrogen-containing tricyclic compounds and drugs containing the same, U.S. Pat. No. 6,316,462 for Methods of inducing cancer cell death and tumor regression, U.S. Pat. No. 6,268,378 for Integrin receptor antagonists, U.S. Pat. No. 6,265,410 for Bicyclic compounds capable of inhibiting tyrosihme kinases of the epidermal growth factor receptor family, for U.S. Pat. No. 6,235,740 Imidazoquinoxaline protein tyrosine kinase inhibitors, U.S. Pat. No. 6,221,900 BTK inhibitors and methods for their identification and use, U.S. Pat. No. 6,211,215 for Heterocyclic compounds, their production and use, U.S. Pat. No. 6,162,613 for Methods for designing inhibitors of serine/threonine-kinases and tyrosine kinases, U.S. Pat. No. 6,147,073 for Substituted tetralymethylen-Oxindoles analogues as tyrosine kinase inhibitors, U.S. Pat. No. 5,990,109 for Heterocyclo-substituted imidazopyrazine protein tyrosine kinase inhibitors, U.S. Pat. No. 5,985,877 for Combination of tyrosine kinase inhibitor and chemical castration to treat prostate cancer, U.S. Pat. No. 5,968,508 for Antagonists to insulin receptor tyrosine kinase inhibitor, U.S. Pat. No. 5,905,149 for Substituted quinolymethylen-oxindole analogues as tyrosine kinase inhibitors, U.S. Pat. No. 5,872,223 for Immunoconjugates comprising tyrosine kinase inhibitors, U.S. Pat. No. 5,789,448 for Benzoylethylene derivative, U.S. Pat. No. 5,719,135 for Substituted 3-arylidene-7-azaoxindole compounds and process for their preparation, U.S. Pat. No. 5,663,346 for Substituted azaindolylidene compounds and process for their preparation, U.S. Pat. No. 5,639,757 for 4-aminopyrrolo[2,3-d]pyrimidines as tyrosine kinase inhibitors, U.S. Pat. No. 5,627,207 for Arylethenylene compounds which are useful as tyrosine kinase inhibitors, U.S. Pat. No. 5,587,385 for Arylidene-heterocyclic derivatives and process for their preparation, U.S. Pat. No. 5,488,057 for 2-oxindole compounds which have useful tyrosine kinase activity, and U.S. Pat. No. 5,374,652 for 2-oxindole compounds which are useful as tyrosine kinase inhibitors, all of which are herein incorporated by reference at least for material related to tyrosine kinase inhibitors.

67. A MEK inhibitor can be any molecule that reduces MEK activity as compared to a control. Inhibitors of MEK include 2-(2-amino-3-methoxyphenyl)4-oxo-4H-[1]benzopyran (PD098059) and U0126. PD098059 is a small molecule that inhibits the activity of MEK 1 and MEK2 via direct noncompetitive binding and results in decreased phosphorylation of MEK 1 and MEK 2 and decreased activation of the MEK substrates ERK1 and ERK2 (U.S. Pat. No. 6,251,943 and Dudley et al., (1995) Proc. Natl. Acad. Sci. 92:7686-7689.). U0126 is a monoclonal antibody specific for MEK. International patent publications WO99/01421 and WO99/01426 are herein incorporated by reference for their teachings on MEK inhibitors and methods of their preparation.

68. Representative examples of MEK inhibitors can be found in U.S. Pat. No. 6,469,004 for Benzoheterocycles and their uses as MEK inhibitors, U.S. Pat. No. 6,440,966 Benzenesulfonamide derivatives and their use as MEK inhibitors, U.S. Pat. No. 6,316,462 Methods of inducing cancer cell death and tumor regression, U.S. Pat. No. 6,251,943 Method of treating or preventing septic shock by administering a MEK inhibitor, and U.S. Pat. No. 6,037,136 for Interactions between RaF proto-oncogenes and CDC25 phosphatases, and uses related thereto, all of which are herein incorporated by reference at least for material related to MEK inhibitors.

69. The Ras protein may also be inhibited. A Ras inhibitor is any molecule that can reduce Ras activity as compared to a control. Farnesyl protein transferase inhibitors such as fused-ring tricyclic bezocycloheptapyridine (e.g., SCH66336) interfere with post-translational processing of Ras proteins (U.S. Pat. No. 6,316,462) thus inhibiting the tyrosine kinase pathway.

70. An exemplary list of United States patents that disclose Ras inhibitors is U.S. Pat. No. 6,414,145 for Imidazolyl compounds as inhibitors of famesyl-protein tranferase, U.S. Pat. No. 6 6,218,375 for Complex of ras-farnesyltransferase inhibitor and sulfobutylether-7-.beta.-cyclodextrin or 2-hydroxypropyl-.beta.-cyclodextrin and method, U.S. Pat. No. 6,103,732 for Carboxylic acid derivatives, their production and use, U.S. Pat. No. 6,087,349 for Method for blocking neoplastic transformation of cells induced by ras oncogenes, U.S. Pat. No. 6,083,985 for Medicinal composition, U.S. Pat. No. 6,037,136 for Interactions between RaF proto-oncogenes and CDC25 phosphatases, and uses related thereto, U.S. Pat. No. 5,571,792 for Histidine and homohistidine derivatives as inhibitors of protein farnesyltransferase, and U.S. Pat. No. 5,567,729 for Farnesyl compounds as farnesyl protein transferase inhibitors to treat ras induced tumor growth, all of which are herein incorporated by reference at least for material related to Ras inhibitors.

71. The disclosed compositions and methods involve signaling through the PI3K/AKT kinase pathway. The PI3K/AKT kinase pathway refers to the signaling pathway which utilizes a PB3K/AKT kinase for signal transduction. An inhibitor of the pathway is any molecule capable of reducing the signal transduction as compared to a control of the PI3K/AKT kinase pathway. PI3K/AKT kinase pathways can involve many different signaling events utilizing many different types of signaling molecules. For example, tyrosine specific protein kinase receptors can be involved. Tyrosine specific protein kinase receptors for this pathway can be involved in the growth and differentiation of cells and can typically be categorized into the following six structural subfamilies: EGF receptors, insulin-receptor/IGF-1 receptors, NGF receptors, PDGF receptor/M-CSF receptors, FGF receptors, and VEGF receptors. The structural differences in these subfamilies and their significance can be determined by sequence and functional comparison. Typically the receptor tyrosine kinases have the common feature of an intracellular kinase domain which can be interrupted by a kinase insert region.

72. Typically upon binding to a receptor tyrosine kinase, receptor dimers (tetramers in the case of IGF-1 receptor and the insulin receptor) formn which can activate the cytoplasmic catalytic domain (intracellular kinase domain) through the cross-phosphorylation of the tyrosine residues. The dimerization (tetramerization in the case of IGF-1 receptor and the insulin receptor) and subsequent phosphorylation of the tyrosine residues is referred to as autophosphorylation. The newly phosphorylated tyrosine residues serve as high affinity binding sites for intracellular signaling proteins (e.g., GTPase activating proteins (GAP), phospholipasse C-γ, and Src-like nonreceptor protein tyrosine kinases), which can subsequently become phosphorylated and activated.

73. PI3K/Akt inhibitors include but are not limited to SH-5 (A.G. Scientific, Inc., San Diego, Calif.); SH-6 (A.G. Scientific, Inc., San Diego, Calif.); IL-6-hydroxymethyl-chiro-inositol 2(R)-2-O-methyl-3-O-octadecylcarbonate (Martelli, A M et al. (2003) Leukemia 17(9): 1794-1805); SR13668; wortmannin and LY294002 Paez and Sellers (2003) Cancer Treat Res. 115:145-67); and API-59 (Tang et al. (2003) 34th Annual Meeting Society of Gynecologic Oncologists: abstract 213). Other PI3k/Akt inhibitors can be found in, for example, U.S. Pat. No. 6,245,754; U.S. Pat. No. 5,053,399; and U.S. Pat. No. 4,988,682 for teachings relating to 3-deoxy-D-myo-inositol ether lipid analogs as inhibitors of PI3k; U.S. Pat. No. 6,187,586 for teachings of antisense modulation of Akt3 expression; U.S. Pat. No. 6,043,090 for teachings of antisense inhibition of Akt2 expression; U.S. Pat. No. 5,958,773 for teachings of antisense modulation of Akt1 expression; and U.S. Pat. No. 6,124,272 for teachings of antisense modulation of PDK-1 expression.

74. Also disclosed are MDM2 inhibitors which can be found in, for example, U.S. Pat. No. 6,399,755 and U.S. Pat. No. 5,858,976. It is understood that general kinase pathway inhibitors can be used to inhibit the MAP kinase pathway, PI3K/Akt pathway, and the MDM2 pathway. Thus specifically contemplated are general kinase inhibitors which can be found in, for example, U.S. Pat. No. 6,495,582 for teachings on Isoxazole; U.S. Pat. No. 6,638,926; U.S. Pat. No. 6,613,776; and U.S. Pat. No. 6,610,677 for teachings relating to Pyrazole; U.S. Pat. No. 6,495,558 for teachings relating to kinase inhibitors, all of which are specifically incorporated herein by reference for at least the molecules they teach and their uses.

### 6. Compositions of Anti-androgens and Kinase Pathway Inhibitors

75. As discussed herein, there are numerous compositions which act as anti-androgens, such as hydroxyflutamide, and numerous compositions that act as inhibitors of kinase pathways. One embodiment is compositions comprising an anti-androgen and a MAP kinase inhibitor. Another embodiment is compositions comprising an anti-androgen and a PI3K/Akt inhibitor. Disclosed are compositions that comprise at least one anti-androgen and one kinase pathway inhibitor. It is understood that these can be in any combination and that multiple representatives of anti-androgens and kinase pathway inhibitors can also be used. As discussed herein, the compositions will typically contain an efficacious amount of both an antiandrogen and a kinase inhibitor. Thus specifically disclosed are compositions comprising at least one anti-androgen and one MAP kinase inhibitor or PI3K/Akt inhibitor. For example, the disclosed compositions can comprise hydroxyflutamide (Flutamide) and U0126. Another example of the disclosed compositions is a compositions comprising hydroxyflutamide and LY294002. It is understood that the disclosed compositions can comprise any combination of an anti-androgen and a kinase inhibitor.

### 7. Characteristics of Nucleic Acid Based Compositions

### a) Sequence Similarities

76. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

77. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence Those of skill in the art readily understand how to determine the homology of two proteins or nucleic acids, such as genes. For example, the homology can be calculated after aligning the two sequences so that the homology is at its highest level.

78. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

79. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

80. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### b) Hybridization/Selective Hybridization

81. The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

82. Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6× SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6× SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and fuirther, depending upon the G-C or A-T richness of any area wherein high homology is desired, all as known in the art.

83. Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their kd, or where only one of the nucleic acid molecules is 10 fold or 100 fold or 1000 fold or where one or both nucleic acid molecules are above their kd.

84. Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

85. Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

86. It is understood that those of skill in the art understand that if a composition or method meets any one of these criteria for determining hybridization either collectively or singly it is a composition or method that is disclosed herein.

### c) Nucleic Acids

87. There are a variety of molecules disclosed herein that are nucleic acid based, including for example the nucleic acids that encode, for example MAP kinase or Ras, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### (1) Nucleotides and Related Molecules

88. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

89. A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

90. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through, a moiety other than a phosphate moiety. Nucleotide substitutes are able to conform to a double helix type structure when interacting with the appropriate target nucleic acid.

91. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

92. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

93. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### (2) Sequences

94. There are a variety of sequences related to for example, the MAP kinase or MEK or Ras or PI3K or Akt or Mdm2 genes, particularly the human homologs of these genes, as well as the other proteins disclosed herein, which can be found in Genbank, these sequences and others are herein incorporated by reference in their entireties as well as for individual subsequences contained therein.

95. It is understood that the description related to this sequence is applicable to any sequence related to the compositions disclosed herein unless specifically indicated otherwise. Those of skill in the art understand how to resolve sequence discrepancies and differences and to adjust the compositions and methods relating to a particular sequence to other related sequences (i.e. sequences of MAP kinase or MEK or Ras or PI3K or Akt or Mdm2 or any other disclosed peptide). Primers and/or probes can be designed for any MAP kinase or MEK or Ras or PI3K or Akt or Mdm2 or any other disclosed peptide sequence given the information disclosed herein and known in the art.

### (3) Primers and Probes

96. Disclosed are compositions including primers and probes, which are capable of interacting with, for example, the MAP kinase or MEK or Ras or PI3K or Akt or Mdm2 or any other disclosed peptide nucleic acids, such as mRNA, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner.

### d) Delivery of the Compositions to Cells

97. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### (1) Nucleic Acid Based Delivery Systems

98. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

99. As used herein, plasmid or viral vectors are agents that transport the disclosed nucleic acids, such as that which encodes MAP kinase or MEK or Ras or PI3K or Akt or Mdm2 or any other disclosed peptide into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

100. Viral vectors can have higher transaction (ability to introduce genes) abilities than chemical or physical methods to introduce genes into cells. Typically, viral vectors contain, nonstructural early genes, structural late genes, an RNA polymerase II transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (a) Retroviral Vectors

101. A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1 985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

102. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

103. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (b) Adenoviral Vectors

104. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

105. A viral vector can be one based on an adenovirus which has had the E1 gene removed and these virons are generated in a cell line such as the human 293 cell line. In another preferred embodiment both the E1 and E3 genes are removed from the adenovirus genome.

### (c) Adeno-asscociated Viral Vectors

106. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

107. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

108. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

109. The disclosed vectors thus provide DNA molecules which are capable of integration into a manimalian chromosome without substantial toxicity.

110. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (d) Large Payload Viral Vectors

111. Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson,.Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems-are also being used to package pieces of DNA>220 kb and to infect cells that can stably maintain DNA as episomes.

112. Other useful systems include, for example, replicating and host-restricted non-replicating vaccinia virus vectors.

(2) Non-nucleic Acid Based Systems

113. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

114. Thus, the compositions can comprise, in addition to the disclosed vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No.4,897,355. Furthermore, the compound can be administered as a component of a miicrocapsule that can be targeted to specific cell types, such as macrophages, or where the diffision of the compound or delivery of the compound from the microcapsule is designed for a specific rate or dosage.

115. In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

116. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, dependig on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

117. Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

118. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### (3) In Vivo/Ex Vivo

119. As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

120. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### e) Expression Systems

121. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (1) Viral Promoters and Enhancers

122. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

123. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Baneiji, J. L. et al., Cell 33: 729 (1983)) as well as within the coding sequence itself(Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

124. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

125. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

126. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

127. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### (2) Markers

128. The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

129. In some embodiments the marker may be a selectable marker. Examples of suitable selectable markers for mammalian cells are dihydrofolate reductase (DHFR), thymidine kinase (TK), neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR cells and mouse LTK cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

130. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### f) Peptides

### (1) Protein Variants

131. As discussed herein there are numerous variants of the MAP kinase or MEK or Ras or PI3K or Akt or Mdm2 or any other disclosed protein that are known and herein contemplated. In addition, to the known fimctional allelic variants there are variants of the MAP kinase or MEK or Ras or PI3K or Akt or Mdm2 or any other disclosed proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from I to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic | Glu |
| acidp | |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.
Alaser
Arglys, gln
Asngln; his
Aspglu
Cysser
Glnasn, lys
Gluasp
Glypro
Hisasn; gln
Ileleu; val
Leuile; val
Lysarg; gln;
MetLeu; ile
Phemet; leu; tyr
Serthr
Thrser
Trptyr
Tyrtrp; phe
Valile; leu

133. Substantial changes in fuinction or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

134. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

135. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

136. Certain post-translational derivatizations are the result of the action of recombinant host cells on the expressed polypeptide. Glutaminyl and asparaginyl residues are frequently post-translationally deamidated-to the corresponding glutamyl and asparyl residues. Alternatively, these residues are deamidated under mildly acidic conditions. Other post-translational modifications include hydroxylation of prpline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

137. It is understood that one way to define the variants and derivatives of the disclosed proteins herein is through defining the variants and derivatives in terms of homology/identity to specific known sequences. For example, there are a number of sequences set forth and sets forth, and specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

138. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

139. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

140. It is understood that the description of conservative mutations and homology can be combined together in any combination, such as embodiments that have at least 70% homology to a particular sequence wherein the variants are conservative mutations.

141. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also-understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

142. It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid-into a peptide chain in a site specific way.

143. Molecules can be produced that resemble peptides, but which are not connected via a natural peptide linkage. For example, linkages for amino acids or amino acid analogs can include CH₂NH—, —CH₂S—, —CH₂—CH₂—, —CH=CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

144. Amino acid analogs and analogs and peptide analogs often have enhanced or desirable properties, such as, more economical production, greater chemical stability, enhanced pharmacological properties (half-life, absorption, potency, efficacy, etc.), altered'specificity (e.g., a broad-spectrum of biological activities), reduced antigenicity, and others.

145. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### D. Methods of Making the Compositions

146. The compositions disclosed herein and the compositions necessary to perform the disclosed methods can be made using any method known to those of skill in the art for that particular reagent or compound unless otherwise specifically noted.

147. The synthesis of the disclosed compositions can be readily accomplished by following established protocols. Furthermore, many of the compositions can be purchased from a variety of sources. Disclosed are processes for making the compositions as well as making the intermediates leading to the compositions.

### 1. Methods of Identifying Inhibitors of Prostate Cancer

148. Disclosed are systems which can be used to identify compounds that affect the MAP kinase pathway which is affected by antiandrogens, such as hydroxyflutamide. There a number of components which are present in these systems. It is understandable that the components are general and that they may be substituted with functional equivalents. One aspect of the systems is that the systems should be able to up-regulate MAP kinase pathway activity. As discussed herein there can be different components to the MAP kinase signaling pathway including tyrosine kinases, Ras, and Raf. The systems can have the various components discussed herein, and in the Examples, expressed in cellular systems which utilize either regulatable or constitutive promoter systems to express the various components.

149. The system also typically will include a means of expressing MAP kinase, MEK, MAP kinase kinase kinase, Ras and/or Raf. This aspect of the systems allows for an MAP kinase pathway that can be monitored for activation.

150. The system then can comprise a variety of components, such as potential inhibitors of the MAP kinase pathway. The systems also typically will be associated with a hydroxyflutamide or analog or other antiandrogen or anti-prostae cancer agent. The systems are typically designed so that activation of the MAP kinase pathway, by for example, hydroxyflutamide can be monitored and molecules can be tested for inhibition in the presence of the activator, such as hydroxyflutamide. Typically the systems will involve controls of either no potential inhibitor or no activation.

151. The systems can also use a variety of cells that express one or more of the components naturally. For example, prostate cancer cells, such as DU145 cells, can be used.

152. Disclosed are cells comprising, any of the proteins disclosed herein. Also disclosed are cells further comprising an inhibitor of a MAP kinase pathway, and/or an inhibitor of prostate cancer, such as an anti-androgen, such as hydroxyflutamide, and/or a potential inhibitor of the MAP kinase pathway.

153. These systems can be used to identify compositions having the desired effects on the MAP kinase pathway in the presence of for example, hydroxyflutamide. For example, compositions which potentially inhibit the MAP kinase pathway activation by hydroxyflutamide as described herein can be assayed for their effect in the system. The systems can be used in a variety of ways as discussed herein.

154. Also disclosed are methods wherein the systems are androgen receptor negative. For example, these can be cells wherein the cells comprise an activatable MAP kinase pathway, but do not express androgen receptor. Also disclosed are systems that can activate the MAP kinase pathway via hydroxyflutamide in the absence of androgen receptor.

2. Compositions Identified by Screening with Disclosed Compositions/Combinatorial Chemistry

### a) Combinatorial Chemistry

155. The disclosed compositions and systems can be used as targets for any combinatorial technique to identify molecules or macromolecular molecules that interact with the disclosed compositions in a desired way. The nucleic acids, peptides, and related molecules disclosed herein can be used as targets for the combinatorial approaches.

156. It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, MAP kinase, MEK, Ras, PI3K, Akt, Mdm2, and other proteins and systems, are also disclosed. Thus, the products produced using the combinatorial or screening approaches that involve the disclosed compositions, such as, MAP kinase, MEK, Ras, PI3K, Akt, or Mdm2, are also considered herein disclosed.

157. Combinatorial chemistry includes but is-not-limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

158. There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

159. A preferred method for isolating proteins that have a given function is described by Roberts and Szostak (Roberts R. W. and Szostak J. W. Proc. NatI. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

160. Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of MAP kinase, MEK, Ras, PI3K, Akt, or Mdm2 is attached to a DNA binding domain of a transcriptional activation protein, such as Gal 4. By performing the Two-hybrid technique on this type of system, molecules that bind the portion of MAP kinase, MEK, Ras, PI3K, Akt, or Mdm2 can be identified.

161. Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are-well known to those of skill in the art.

162. Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

163. Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

164. Molecules isolated which can either be competitive inhibitors or non-competitive inhibitors of the hydroxyflutamide activation of the MAP kinase pathway are disclosed, and can be identified using the disclosed methods.

165. In another embodiment the inhibitors are non-competitive inhibitors of the Map kinase pathway activated by, for example hydroxyflutamide.

166. As used herein combinatorial methods and libraries included traditional screening methods and libraries as well as methods and libraries used in interative processes.

### b) Computer Assisted Drug Design

167. The disclosed compositions can be used as targets for any molecular modeling technique to identify either the structure of the disclosed compositions or to identify potential or actual molecules, such as small molecules, which interact in a desired way with the disclosed compositions.

168. It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, MAP kinase, MEK, Ras, PI3K, Akt, or Mdm2 and other disclosed proteins and systems, are also disclosed. Thus, the products produced using the molecular modeling approaches that involve the disclosed compositions, such as, MAP kinase, MEK, Ras, PI3K, Akt, or Mdm2, are also considered herein disclosed.

169. Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

170. Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

171. A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activitv Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

172. Although described above with reference to design and generation of compounds which could alter binding, one could also screen libraries of known compounds, including natural products or synthetic chemicals, and biologically active materials, including proteins, for compounds which alter substrate binding or enzymatic activity.

### E. Methods of Using the Compositions

### 1. Method of Treating Cancer

173. The disclosed compositions can be used to treat any disease where uncontrolled cellular proliferation associated with androgen receptor occurs. For example, the disclosed compositions can be used for treating prostate cancer.

174. The compositions can be administered as disclosed herein, or using any effective means.

175. Disclosed are methods of inhibiting prostate cancer comprising administering any of the compositions discussed herein, as well as compositions identified by the methods disclosed herein.

176. Also disclosed are methods of administering a MAP kinase, MEK, Ras, PI3K, Akt, or Mdm2 pathway inhibitor, after the administration of an anti-prostate cancer compound, such as an anti-androgen, such as hydroxyflutamide. It is understood that the administration of an anti-prostate cancer compound, such as an anti-androgen, such as hydroxyflutamide can cause an activation of the disclosed pathways and that this can be associated with the refractory response prostate cancer patients can undergo when being treated with an anti-prostate cancer compound, such as an anti-androgen, such as hydroxyflutamide. As disclosed herein the MAP kinase, MEK, Ras, PI3K, Akt, or Mdm2 pathway inhibitor and the an anti-prostate cancer compound, such as an anti-androgen can be administered together meaning at the same time or effectively at the same time, i.e. such that there is at least some of each type of inhibitor in the patient for at least some period of time together. The MAP kinase pathway inhibitor can also be added at anytime after the administration of the an anti-prostate cancer compound, such as an anti-androgen, such as hydroxyflutamide, including when the an anti-prostate cancer compound, such as an anti-androgen, such as hydroxyflutamide is no longer being administered and, for example, is no longer effectively active in the patient. Thus, the MAP kinase pathway inhibitor can be administered, at least 1 minute, 5 minutes, 10 minutes, 15 minutes, 30 minutes, 45 minutes, 1 hour, 2 hours, 4 hours, 8 hours, 24 hours, 2 days, 4 days, 10 days, 30 days, 2 months, 4 months, 6 months, 1 year, or 2 years or more after the administration of an antiandrogen, such as hydroxyflutamide.

177. Also disclosed are methods of administering a MAP kinase, MEK, Ras, PI3K, Akt, or Mdm2 pathway inhibitor, before the administration of an anti-prostate cancer compound, such as an anti-androgen, such as hydroxyflutamide. Thus, the MAP kinase pathway inhibitor can be administered, at least 1 minute, 5 minutes, 10 minutes, 15 minutes, 30 minutes, 45 minutes, 1 hour, 2 hours, 4 hours, 8 hours, 24 hours, 2 days, 4 days, 10 days, 30 days, 2 months, 4 months, 6 months, 1 year, or 2 years or more before the administration of an antiandrogen, such as hydroxyflutamide.

178. Disclosed herein is the knowledge that there are prostate cancers caused by later activated MAP kinase pathways, precipitated by the administration of antiandrogens, such as hydroxyflutamide. It is understood that as these cancers arise, the administration of MAP kinase pathway inhibitors can be administered.

### 2. Pharmaceutical Carriers/Delivery of Pharmaceutical Products

179. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in tie subject, as would be well known to one of skill in the art.

180. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

181. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

182. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem. 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

183. The compositions, including antibodies, can be used therapeutically in combination with a pharmaceutically acceptable carrier.

184. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

185. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

186. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

187. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

188. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

189. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

190. Compositions for oral administration include powders or granules, suspensions or solutions in water or non-aqueous media, capsules, sachets, or tablets. Thickeners, flavorings, diluents, emulsifiers, dispersing aids or binders may be desirable.

191. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

192. Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

193. Following administration of a disclosed composition for treating, inhibiting, or preventing an prostate cancer cell growth, the efficacy can be assessed in various ways. For instance, a composition disclosed herein is efficacious in treating or inhibiting prostate cancer in a subject by observing that the composition reduces the number of tumor cells. The number of tumor cells can be measured by, for example, performing a biopsy. The efficacy of the compositions can also be determined by assaying for the prostate specific antigen or PSA, using any technique.

194. The compositions that inhibit prostate cancer and/or cancer cell proliferation disclosed herein may be administered prophylactically to patients or subjects who are at risk for prostate cancer.

### F. EXAMPLES

195. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Activation of MAP Kinase Pathway by the Antiandrogen Hydroxyflutamide in Androgen Receptor Negative Prostate Cancer Cells

### a) Methods

### (1) Immunocytochemistry

196. Immunocytochemical staining were performed on formalin-fixed, paraffin-embedded tissue sections using antibodies ERK1,and ERK2 (SC-94, and SC-154, 1/400 dilution), and phospho-ERK1/2 (SC-7383, 1/50 dilution) all from Santa Cruz (Santa Cruz, Calif.). Sections were cut at 4 to 5 microns and deparaffinized according to established procedures and quenched with 3% hydrogen peroxide for 6 min. Antigen unmasking with heat retrieval in citrate buffer/pH 6.0 was accomplished by placing slides in a microwave (1500 watts) pressure cooker for 30 minutes. Slides were rinsed and stained for 45 minutes with primary antibody, and then incubated for 20 min with secondary antibody and streptavidin-HRP. Slides were developed with AEC+, rinsed and counterstained with Mayer Hematoxylin Blue in 0.3% ammonia water.

### (2) Transient Transfection Assay

197. DU145 cells were obtained from American Type Culture Collection and cultured in Dubecco's modified Eagles's medium (DMEM) supplemented with 10% fetal bovine serum (FBS). Cells (1×10⁵) were seeded in 35 mm plates and transfected with the SuperFect transfection reagent (Qiagen, Chatworth, Calif.). After 4 hours the media were changed to serum-free DMEM media for 24 hours. Thereafter, the cells were treated with HF, EGF, and vehicle for 18 hours and then lysed for luciferase assay. Luciferase activity was normalized for taansfection efficiency using pRL-TK as an internal control. Luciferase assays were performed using dual-luciferase reporter system (Promega, Madison, Wis.).

### (3) Immunoblot of Phosphorylated p44/42 MAP Kinase

198. This assay uses a polyclonal antibody specific against the activated p44/42 MAP kinases (Cell Signaling Technology, Beverly, Mass.), which detects p44 and p42 MAP kinase (ERK1 and 2) only when catalytically activated by dual phosphorylation at Thr202 and Tyr204. DU145 cells were seeded at 1×10⁵ cells per 100 mm plate and allowed to attach overnight and then the media were replaced with serum-free DMEM media for 24 hours. The cells were pre-treated with MAP kinase inhibitors, EGFR antibodies, or cycloheximide for 1 hour and followed by HF, EGF, or vehicle treatment. Cells were washed twice with PBS, harvested at indicated times, and lyzed with RIPA buffer for 30 min on ice. The protein concentrations were determined by Bradford assay, and equal amount of protein was resolved by SDS-PAGE. Gels were transferred, immunoblotted with p44/42 MAP kinase antibody (1/1000), and then incubated with secondary antibody. Proteins were visualized by the enhanced chemiluminescence system (NEN™ Life Science Products, Boston, Mass.). P44/42 MAP kinase (total ERK1/2) was blotted as a control.

### (4) Cell Proliferation Assay

199. DU145 cells were seeded at 2×10⁴/ml on 35 mm plates and allowed to attach overnight. Cells were kept in serum-free DMEM medium for 24 hours, and then media were replaced with DMEM-0.5% FBS and treated as indicated. Cells were trypsinized and counted with a hemacytometer at different times after treatments. For the antisense oligonuleotide experiments, cells were incubated with a fixed ratio of oligonucleotide versus SuperFect (2.5 μl of SuperFect per 100 nM oligo) for 4 hours and then the oligo-containing medium was replaced with DMEM-0.5% FBS. Cells were trypsinized and counted by hemacytometer at indicated times.

### (5) Immunoprecipitation

200. Serum-stared and treated DU145 cells were lysed with RIPA buffer for 30 min on ice. Lysates were then centrifuiged at 12,000 g for 10 min at 4° C. and protein concentrations were determined by the Bradford assay. 500 μg of cell lysates were incubated for 4 hours with either anti-Ras or anti-EGFR antibody (1/100, Santa Cruz Biotechnology) to the protein-antibody mixture and then added in 20 μl of protein A/G plus-agarose (Santa Cruz Biotechnology) for another 4 hours incubation with constant rotation. The immunoprecipitates were washed four times with cold PBS, resolved by SDS-PAGE, and immunoblotted by anti-Raf-1 antibody, or anti-phospho-tyrosine monoclonal antibody (Oncogene Research, Calbiochem, La Jolla, Calif.). The results were visualized by chemiluminescence.

### b) Results

### (1) Increase of Phospho-ERK1/2 Level in the Patients' Tumors After Patients Developed Flutamide Withdrawal Syndrome

201. To evaluate the role activation of MAP kinase plays in the HF withdrawal form of prostate cancer, four prostate cancer patients undergoing androgen ablation therapy with flutamide were examined. Their phosph-ERK1/2 levels in prostate cancer biopsies were compared before and after development of the flutamide withdrawal syndrome. Each slide was examined and scored the number of the cells stained positive for phosph-ERK1/2 under the 100× high power field. It was found that phosphorylated or activated MAP kinase was undetectable before androgen ablation therapy with flutamide treatment (FIG. 1C). In contrast, 30%, 41%, 52% and 45% of the cells were stained positive for phospho-ERK1/2 in the recurrent tumors of all four patients whose disease was progressing while receiving flutamide and who developed flutamide withdrawal syndrome when the medication was discontinued immediately after the second biopsy (FIG. 1D). Mayer hematoxylin blue staining for pathological morphologic examination is shown in FIGS. 1A and B.

### (2) Activation of MAP Kinase Pathway by HF in DU145 Cells

202. To investigate whether HF has any effect on the MAP kinase signal transduction pathway, western blotting was performed using an antibody that specifically recognizes dually phospho-MAP kinase (phospho-ERK1/2) in AR negative DU145 human prostate cancer cells. The results showed that HF has no influence on total MAP kinase (ERK1/2) protein expression. HF at 1 μM can activate MAP kinase within 15 minutes and reach maximum activation in 30 minutes (FIG. 2A lanes 2 and 4). DU145 cells (passage number 61-65, ATCC, HTB-81) is documented as an AR-negative cell line. This is also demonstrated in our FIG. 2B, which shows no visible AR band using AR antibody NH27. The data in FIG. 1A-1B demonstrates that HF can activate MAP kinase via a non-AR-mediated mechanism.

203. It was found that U0126, a specific MAP kinase kinase (MEK) inhibitor, but not U0124, a structurally similar compound without inhibiting effects, can block HF-mediated MAP kinase activation. (FIG. 2C, lanes 7 vs. 9 and 8).

204. For comparison, it was also found that HF-mediated MAP kinase activation could be detected in other selected prostate cancer cells with endogenous AR expression. For example, HF activates MAP kinase in the CWR22, and PC-3 stably transfected with AR (PC3-AR2) (FIGS. 2D and E), but not LNCaP (data not shown). It is unclear whether endogenous AR in these cells plays any roles for the activation of MAP kinase pathway, as the data demonstrate that HF can activate MAP kinase pathway in AR negative cells such as DU145 cells. It was concluded that AR might not be a determining factor to mediate the activation of MAP kinase by HF. The activation of MAP kinase by HF is relatively weak as compared to EGF activation of MAP kinase. Nevertheless, as the concentration of HF needed to activate the MAPK is much lower than the concentration commonly available in the treatment of prostate cancer, the data indicates that HF at pharmacological concentrations (10⁻⁶ to 10⁻⁵ M) can become a potent activator to stimulate MAP kinase pathway. In order to ensure the HF that was used indeed exerts an antiandrogenic effect, the AR/DHT mediated PSA transcriptional activity was examined by adding HF. As shown in FIG. 2F, 1 μM of HF represses DHT induced PSA-Luc activity. Together, data from FIG. 2 suggest that HF can activate the MAP kinase pathway within 15 minutes without involvement of the AR-mediated mechanism.

### (3) HF-mediated MAP Kinase Activation is Via Ras and Raf Pathway

205. To assess whether Ras and Raf are upstream regulators of HF-mediated MAP kinase activation, a Ras-Raf immunoprecipatation assay was employed. As shown in FIG. 3A lanes 2 vs 3 and 4 vs 3. Raf was detected in the Ras immunocomplex in lysates from cells treated with HF or EGF, but not in lysates from cells treated with 9 cis-retinoic acid. These data demonstrate that HF promotes the association between Ras and Raf that may result in the activation of their downstream MAP kinase. Transfection with a dominant negative H-Ras mutant, N17 (Ras-N17), further proved this observation. The phospho-ERK1/2 levels in HF-treated or EGF-treated cells were decreased with co-transfection of Ras-N17 into DU145 cells, in a dose-dependent manner (FIG. 3B, lane 5, 6, 7, 3, and 4), but not in ethanol-treated cells (lane 1 and 2). The optical density (OD) of phospho-ERK1/2 were scanned and quantified by Versa Doc Imaging System and Quantity One software.

### (4) An EGFR Inhibitor and Its Neutralized Antibody Can Inhibit HF-mediated Activation

206. The involvement of the EGF receptor (EGFR) in the upstream events for the HF-mediated Ras-Raf-MAP kinase pathway. Preincubation of DU145 cells with 100 nM of Tyrphostin AG 1478, a selective inhibitor of the EGFR's tyrosine kinase was investigated for 1 hour and results in decreased levels of phospho-ERK1/2 induced by either HF or EGF (FIG. 4A lanes 2 vs. 5 and 3 vs. 6).

207. Ten ng/ml cycloheximide, a protein synthesis inhibitor, failed to block MAP kinase activation mediated by HF or EGF (lanes 8 and 9). However, a slight increment was observed when ethanol was added to cycloheximide treatment (lane 7). These data strongly suggest that the activation of MAP kinase could be a non-genomic effect and did not involve new protein synthesis.

208. Treatment of DU145 cells with monoclonal antibody EGFRmAb-528 or polyclonal antibody EGFRpAb-1005 for 1-hour also abrogated activation of phospho-ERK1/2 induced by either EGF or HF (FIG. 4B) in a dose-dependent manner (lane 3 to 5). Because these antibodies have been demonstrated to bind to a cell surface epitope of the EGFR and to antagonize ligand stimulated EGFR tyrosine kinase activity, the data suggest that RF's activation of the MAP kinase pathway could be exerted through a membrane receptor tyrosine kinase-mediated pathway, without involvement of new protein synthesis in an AR deficient environment. Whether the EGFR itself or an EGFR isoform, which can be recognized by EGFR antibodies, served as the membrane mediator for HF's activation of the Ras-Raf-MAPK pathway remains unclear.

209. The 170 kDa EGFR exercises its biological effects in response to binding of specific polypeptide ligands, including EGF and TGFα. This leads to activation of EGFR catalytic tyrosine kinase domain, autophosphorylation of specific residues in its carboxyl terminus, and recruitment and phosphorylation of signaling proteins. As shown in FIG. 4C, HF causes the autophosphorylation of EGFR as detected by the immunoprecipitation with an antibody to the EGFR and immunoblotting with anti-phosphotyrosine. However, AG1478 can inhibit both EGF- and HF-mediated EGFR autophosphorylation, without affecting total EGFR concentration.

### (5) HF Promoted the Cell Proliferation and Cyclin D1 Expression

210. Because the activation of MAP kinase may result in cell proliferation, it was determined whether HF treatment affected cell proliferation. It was found that HF, like EGF, can promote cell proliferation in a low serum (0.5% FBS) environment after serum starvation (FIG. 5A) significantly. In contrast, if the cells were maintained in 10% FBS medium, HF or EGF causes no significant stimulation of cell proliferation (data not shown). To further link cell proliferation with the HF-mediated activation of Ras/MAP kinase pathway, the DU145 cells were transfected with anti-sense of Ras, Raf and HIV as a control anti-sense. As expected, cells transfected with anti-sense of Ras and Raf oligomers that block the activation of MAP kinase, did not show any significant increase of cell number after 24 hour HF- or EGF-treatment. However, the cells doubled in number when we transfected control anti-sense oligomers and treated with HF or EGF (FIG. 5B). In order to confirm the efficacy of the antisense treatments, endogenous Ras and Raf expression were examined upon the antisense transfection. The endogenous Ras and Raf expression were reduced significantly in the Ras and Raf antisense transfected cell lysate compared with the liposome transfected control. However, the endogenous Ras and Raf expression remained the same when HIV antisense was transfected. In conclusion, it was demonstrated that HF activated the MAP kinase pathway and promoted cell proliferation. Blocking of Ras and Raf can reverse proliferation mediated by HF and EGF. These data provide evidence that HF, like EGF, promotes cell proliferation through the Ras-Raf-MAPK pathway.

211. To further dissect the mechanism of how HF, like EGF, promotes cell proliferation, several potential G1- or S-phase targets that might be influenced by HF or EGF were tested. It was found that cyclin D1 is induced after HF- or EGF-treatment (FIG. 6A, lane 1 vs 2; lane 1 vs 3). In contrast, there is no significant change in other cell cycle related gene products including such as p27, p21, Ki67, and PCNA. The HF- or EGF- mediated cyclin D1 gene expression was confirmed by a cyclin D1 promoter study. A cyclin D reporter construct which contains the human cyclin D promoter, from aa −1745 to +1, linked to the Luciferase reporter (Lin, S.-Y., et al., Proc. Natl. Acad. Sci. USA, 97: 4262-4266, 2000) was used. As shown in FIG. 5B, 10⁻⁶ M HF or 30 ng/ml EGF induced cyclin D1 promoter (−1745D1-Luc) activity about 2-fold (lane 1 vs 2). Importantly, cotransfection of dominant negative of Ras and Raf attenuated this HF- or EGF-mediated induction. An increase in cyclin D1 concentration may trigger transition from G1- to S-phase, and eventually result in increased cell proliferation. The expression of cyclin D1 could be induced by HF further clarifies how HF promotes DU145 cell growth.

212. Flutamide was the first androgen receptor blocker to achieve widespread use. It is metabolized into hydroxyflutamide, the biologically active form of the drug. In 1993, Kelly and Scher described four patients with progressive metastatic prostate cancer combined androgen blockade therapy. After selective discontinuation of flutamide treatments the patients showed a biochemical, and objective improvement (Kelly, W. K. and Scher, H. I., J Urol., 149: 607-609, 1993.). This phenomenon has also been reported for cyproterone acetate (Sella, A., et al., Urology 52: 1091-1093, 1998), nilutamide (Huan, S. D., et al., Urology 49: 632-634, 1997), and bicalutamide (Small, E. J. and Carroll, P. R., Urology 43: 408-410, 1994), as well as for progestational agents (Dawson, N. A. and Mcleod, D. G., J Urol 153: 1946-1947, 1995). It is apparent that prolonged therapy with flutamide may select for tumor cells that are stimulated by hydroxyflutamide and thus contribute to prostate cancer progression. Discontinuation of treatment with stimulatory antiandrogens results in a related withdrawal syndrome. The pathophysiology of antiandrogen withdrawal syndrome is not completely understood, although AR gene mutations seem to be the part of the explanation. However, the transient and incomplete response of the tumor to antiandrogen withdrawal, as well as failure of the withdrawal syndrome to occur in many patients, implies there are mechanisms other than AR mutations that contribute to tumor progression. The progression of prostate cancer to androgen-independent disease is also associated with the elevation and autocrine production of multiple polypeptide growth factors (Culig, Z., et al., Prostate, 28: 392-405, 1996, Culig, Z., et al., Cancer Res., 54: 5474-5478, 1994.). It is widely suspected that the paracrine and autocrine loops that exist play an important role in the loss of hormone dependence, as well as in the dependent of metastatic potential. The growth factors and receptors associated with prostate cancer progression regulate cell growth, at least partly, through regulation of the activity of Ras family members. Ras, a proto-oncogene, is dependent on protein-protein interactions to cause its ultimate dissociation from GDP. The dissociation renders Ras free to bind to GTP and initiate a complex signaling cascade that leads to the activation of the MAP kinases ERK1 and ERK2. Several small molecular inhibitors such as growth factors or their receptors that target specific steps of the MAP kinase cascade have recently entered the clinical arena (Peng, D., et al., Cancer Res., 56: 3666-3669, 1996, Pietrzkowski, Z., et al., Cancer Res. 53: 1102-1106, 1993, Putz, T., et al., Res. 59: 227-233, 1999, and Fong, C-J., et al., Cancer Res. 52: 5887-5892. 1992).

213. Disclosed herein it was demonstrated that HF can have dual roles in the modulation of prostate tumor growth and this data is summarized in FIG. 7. First, when prostate cancer patients have relatively high concentrations of androgens, HF can function as an effective antiandrogen, competing with androgens for binding to the AR. The effect is inhibition of androgen-mediated prostate cancer growth. At later stages, when prostate tumors become androgen independent, the continuation of HF-treatment triggers MAP kinase pathway activation, with subsequent stimulation of prostate tumor growth. This phenomenon is consistent with the flutamide withdrawal syndrome and can explain why in the case of metastatic carcinoma, little prolongation of survival can be demonstrated for combined androgen blockade (simultaneous administration of castration and antiandrogen therapy) compared with androgen deprivation monotherapy. Moreover, this observation offers potential therapeutic targets that may prolong the antitumor effect of flutamide.

### 2. Example 2

### Suppression vs. Induction of Androgen Receptor Functions by the Phophatidylinositol 3-kinase/Akt Pathway in Prostate Cancer LNCaP Cells with Different Passage Numbers

### a) Materials and Methods

### (1) Reagents

214. pCDNA3 cAkt was previously described (Lin, H. K., et al. (2001) Proc Natl Acad Sci U S A 98, 7200-7205) and mtAR S210A/S790A was described (Lin, H. K., et al. (2002) Embo J 21, 4037-4048). pCDNA3-PTEN was a gift from Dr. Charles L. Sawyers and pGEX-KG-PTEN was from Dr. Frank B. Furnari. IGF-1 and LY294002 was from Calbiochem. 5α-dihydrotestosterone (DHT), doxycycline (Dox), and cyclohexamide were from Sigma. The anti-AR polyclonal antibody, NH27, was produced as previously described (Lin, H. K., et al. (2001) Proc Natl Acad Sci USA 98, 7200-7205). The mouse monoclonal PTEN and PSA antibodies and the goat polyclonal β-actin antibody were from Santa Cruz. The mouse monoclonal Akt and phospho-Akt (S473) antibodies were purchased from Cell Signaling.

### (2) Cell Culture and Transfections

215. The DU145, PC-3, and COS-1 cell lines were maintained in Dulbecco's Minimum Essential Medium containing penicillin (25 U/ml), streptomycin (25 μg/ml), and 10% fetal calf serum (FCS). The LNCaP cells were maintained in RPMI-1640 with 10% FCS. Transfections were performed using SuperFect™ according to standard procedures (Qiagen).

### (3) Luciferase Reporter Assays

216. Luciferase reporter assay was described previously with some modifications (Hu, Y. C., et al. (2002) J Biol Chem 277, 33571-33579). The cells were transfected with plasmids in 10% charcoal stripped serum (CSS) media for 16 h and then treated with ethanol or 10 nM DHT for 16 h. The cells were lysed and the luciferase activity was detected by the dual luciferase assay according to standard procedures (Promega). Mouse mammary tumor virus-luciferase (MMTV-luc), which contains the AR response elements, was used as an AR transactivation reporter. The results were normalized by renilla luciferase activity (pRL-SV40-luc) and the data are represented as means±s.d. from triplicate sets of three independent experiments.

### (4) LNCaP Stable Transfectants

217. For the Dox-inducible system, PTEN was released from pGEX-KG-PTEN using EcoRI digestion and inserted into pBIG2i vector. The LNCaP cells were transfected with pPIB2i PTEN for 24 h. The cells were selected using 100 μg/ml hygromycin. Individual single colonies were picked and grown until 70% confluent, followed by 4 μg/ml Dox treatment for 48 h. The positive clones were confirmed by Western blot analysis.

### (5) Generation of an Anti-phospho (S210) AR Antibody

218. The phospho-AR peptide (SGRAREADGAPTSSKD) was generated and used for generation of anti-phospho-AR (S210) antibody (clone 156C135.2) according to the manufacture's procedures (AndroScience, San Diego, Calif.).

### (6) Immunoprecipitation and Western Blot Analysis

219. The imrnunoprecipitation and Western blotting were performed as previously described (Lin, H. K., et al. (2001) Proc Natl Acad Sci USA 98, 7200-7205). The cell extracts (1 mg) were immunoprecipitated with the indicated antibody. The immunocomplexes were subjected to 8% SDS-PAGE and immunoblotted with the indicated antibody.

### (7) Cell Growth Assay

220. LNCaP cells (2×10⁴) with different passage numbers were grown in 12-well plates, transfected with parent vector or cAkt, and cultured in the 10% CSS media after 3 h transfection. Cells were stained by trypan blue on different days, as indicated, and cell numbers were determined by direct counting on hemacytometers. The data are represented as means±s.d. from triplicate sets of three independent experiments.

### b) Results and Discussion

### (1) The Cell-specific and Passage-dependent Effect of PI3K/Akt Signaling on AR Activity

221. The PI3K/Akt pathway plays an important role in cell growth, survival, adhesion and migration in a variety of cell types. In the prostate cancer LNCaP cells, the PI3K/Akt pathway is a dominant survival signal pathway for cells and inhibition of this pathway by P13K inhibitors leads to cell growth arrest and apoptosis (Lin, J., et al. (1999) Cancer Res 59, 2891-2897). Recently, it has been demonstrated that the PI3K/Akt pathway could regulate AR activity via inducing its phosphorylation (Lin, H. K., et al. (2001) Proc Natl Acad Sci U S A 98, 7200-7205; Wen, Y., et al. (2000) Cancer Res 60, 6841-6845). While activation of the PI3K/Akt pathway suppresses AR activity in androgen-independent prostate cancer DU145 cells (Lin, H. K., et al. (2001) Proc Natl Acad Sci USA 98, 7200-7205), other reports also demonstrated that the PI3K/Akt pathway enhances AR activity in androgen-dependent prostate cancer LNCaP cells (Wen, Y., et al. (2000) Cancer Res 60, 6841-6845; Li, P., et al. (2001) J Biol Chem 276, 20444-20450). Although the detailed mechanism of these differential effects remains unclear, it is possible that different cell types can have differential PI3K/Akt effects on AR activity.

222. Interestingly, it was found that the PI3K/Akt pathway could regulate AR activity in a passage-dependent manner in LNCaP cells. Constitutively active form of Akt (cAkt) suppressed AR activity in low passage number LNCaP cells (passage number 25 (P25)) (FIG. 8A), but enhanced AR activity in high passage number LNCaP cells (P60) (FIG. 8B), in reporter gene assays. It should be noted that the reporter gene activation by androgen was much higher in higher passage LNCaP cells (compare FIG. 8B with FIG. 8A). This can indicate that some factors that preferentially exist or are over-expressed in higher passage LNCaP cells can contribute to enhancing this androgen response. Blockage of the PI3K/Akt pathway by LY294002 slightly enhanced AR activity in low passage number LNCaP cells, but suppressed AR activity in high passage number LNCaP cells (FIGS. 8A and B, lane 4). Although LY294002 has been widely used as a PI3K inhibitor, at 20 tM this reagent can affect other kinases that influence the AR activity. Western blot assays were performed to examine the role of the PI3K/Akt pathway in regulating AR target gene expression. Even though LY294002 only marginally enhanced AR activity in low passage LNCaP cells in the reporter gene assays (FIG. 8A), it increased androgen-induced prostate specific antigen (PSA) expression, an AR target gene, in low passage number LNCaP cells (FIG. 8C). Similar to the reporter gene assay, LY294002 suppressed PSA expression in high passage number LNCaP cells (FIG. 8C). Moreover, cAkt reduced androgen-induced PSA expression in low passage number LNCaP cells, but slightly enhanced PSA expression in high passage number LNCaP cells (FIG. 8D). These results indicate that distinct passage numbers of LNCaP cells might influence the effects of the PI3K/Akt effect on AR activity. Using PC-3 cells, Thompson et al. also demonstrated that the PI3K/Akt pathway could suppress AR activity (Thompson, J., et al. (2002) TheEndocrine Society P3-141, 526), which is consistent with the data (FIG. 8A) and early reports using DU145 cells as the cell model (Lin, H. K., et al. (2001) Proc Natl Acad Sci U S A 98, 7200-7205). Together, these results demonstrate that the effects of the PI3K/Akt signaling pathway on AR activity can change with different prostate cancer cell lines and with the same cell line at different passage numbers.

223. At early stages, prostate cancer cells need androgen signaling for growth and survival. Androgen ablation or anti-androgen treatment can lead to cell growth arrest and apoptosis of these androgen-sensitive cancer cells (Carson, J. P., et al. (1999) Cancer Res 59, 1449-1453). The basal activity of the PI3K/Akt pathway in the early-stage prostate tumors is lower and is not adequate to play major roles in maintenance of prostate e cancer cell growth and survival in the absence of concurrent androgen signaling. However, androgens can become less important factors for tumor cell growth and survival in late-stage prostate cancer. In contrast, tumor cells at this later stage have higher basal activity of the PI3K/Akt pathway that can contribute to the development of prostate cancer progression by preventing cells from apoptosis (Graff, J. R., et al. (2000) J Biol Chem 275, 24500-24505).

224. It was found that the low passage LNCaP cells possess a low basal level of Akt activity (FIG. 8E). In contrast, high passage LNCaP cells show a strong basal Akt activity (FIG. 8E). The data show that Akt negatively modulates AR activity in low passage LNCaP cells (FIG. 8A), indicating that LNCaP cells at this early stage require more androgen to compensate for the suppressive effect of the low basal Akt activity, and that the low basal Akt activity is not sufficient to provide the survival signal necessary for maintenance of cell growth and survival.

225. To determine whether Akt is a determining factor for the androgen reliance of LNCaP cell growth, LNCaP cells were cultured in CSS media lacking the androgen to compare the growth pattern of LNCaP cells at different passage numbers in the presence or absence of cAkt. Early passage LNCaP cells, with the low basal activity of Akt, showed little cell growth in the CSS media (FIG. 8F), indicating the androgens are important for cell growth. In contrast, high passage LNCaP cells, with higher basal Akt activity, grew much faster than early passage LNCaP cells (FIG. 8F), indicating less dependence on the androgens. Elevation of the basal Akt activity by transfection of cAkt significantly increased the LNCaP cell growth at both cell passages, albeit the effect of cAkt was more profound in the early passage LNCaP cells (FIG. 8F). Thus, the Akt signal can be a key factor in driving LNCaP cell growth and survival at this late stage with weaker androgen signaling.

226. Considering the biphasic effect of PI3K/Akt and androgen signaling on progression of prostate cancer, androgen ablation therapy, which removes most of the androgens available to prostate tumors, can result in increased activation of the PI3K/Akt pathway, promoting tumor cell growth and survival. This is further supported by a recent report showing that the PI3K/Akt pathway is elevated in LNCaP cells cultured in androgen-depleted medium (Murillo, H., et al. (2001) Endocrinology 142, 4795-4805). Increased PI3K/Akt signaling upon loss of androgen signaling can contribute to the failure of androgen ablation therapy at later stages of prostate cancer. For this reason, use of combination therapy that includes androgen ablation at early stages and suppression of the PI3K/Akt pathway at later stages can provide a better strategy to battle prostate cancer.

### (2) The Effect of PI3K/Akt Signaling on AR Phosphorylation

227. AR is a phosphoprotein and its activity can be modulated by phosphorylation (Heinlein, C. A., et al. (2002) Endocr Rev 23, 175-200). Previously, it was demonstrated that activation of PI3K/Akt pathways by insulin-like growth factor-1 (IGF-1), in COS-1 cells, induces AR phosphorylation in vivo (Lin, H. K., et al. (2001) Proc Natl Acad Sci U S A 98, 7200-7205). The in vitro kinase assay-further revealed that Akt, but not PI3K, phosphorylates AR at Serine 210 (S210) and S790 residues that are the Akt consensus phosphorylation sites (Lin, H. K., et al. (2001) Proc Natl Acad Sci U S A 98, 7200-7205). Overexpression of cAkt, but not a kinase-dead Akt mutant (dAkt), induced AR phosphorylation in vivo, and mutations at the consensus serine residues reduced Akt-mediated AR phosphorylation (Lin, H. K., et al. (2001) Proc Natl Acad Sci USA 98, 7200-7205). Consistent with these results, Wen et al. also found that Akt associated with AR and phosphorylated AR at S210 and S790 in vitro (Wen, Y., et al. (2000) Cancer Res 60, 6841-6845).

228. To determine whether gene overexpression was a confounding factor in the interpretation of the AR phosphorylation assays, IGF-1 was used to activate endogenous PI3K/Akt and therefore mimic physiological conditions. FIG. 9A, demonstrates that IGF-1 treatment induced AR phosphorylation in LNCaP cells (P38) and adding the PI3K inhibitor LY294002 blocked IGF-1-mediated AR phosphorylation, indicating that the PI3K/Akt pathway is involved in the phosphorylation of AR. Using a site-specific anti-phosphoserine AR antibody, AR phosphorylation at S210 was detected when LNCaP cells were treated with IGF-1 (FIG. 9B). Moreover, using the Dox-inducible system the inducible PTEN clone, a tumor suppressor that antagonizes the PI3K/Akt pathway (Di Cristofano, A., et al. (2000) Cell 100, 387-390), in LNCaP cells at P40 was generated. PTEN expression induced by Dox treatment inhibited Akt activation and AR phosphorylation at S210 (FIG. 9C). IGF-1 also induced wild-type (wtAR) phosphorylation in COS-1 cells (FIG. 9D), and LY294002 blocked the IGF-1-mediated phosphorylation. In contrast, IGF-1 did not induce phosphorylation of mutant AR (mtAR) (S210A/S790A) in which two Akt consensus sites were mutated from serine (S) to alanine (A) (FIG. 9D). These data therefore strongly support the earlier findings that the PI3K/Akt pathway activated by IGF-1 mediates AR phosphorylation at S210 and S790 (Lin, H. K., et al. (2001) Proc Natl Acad Sci U S A 98, 7200-7205). Therefore the level of the Akt activity in LNCaP cells is not sufficient to induce AR activity, given that the basal level of Akt activity is low in early passage LNCaP cells (FIG. 8E), in which AR phosphorylation by Akt may not occur and can require addition of growth factors to amplify the PI3K/Akt signal.

### (3) Regulation of AR Protein Turnover by the PI3K/Akt Pathway

229. Growing evidence implies that AR may be degraded by the ubiquitin-proteasome pathway (Yeh, S., et al. (2000) Proc Natl Acad Sci U S A 97, 11256-11261; Poukka, H., et al. (2000) J Cell Sci 113, 2991-3001; Sheflin, L., et al. (2000) BiochemBiophys Res Commun 276, 144-150). In support of this notion, it was recently demonstrated that activation of the PI3K/Akt pathway induces AR ubiquitylation and subsequent degradation by the 26S proteasome (Lin, H. K., et al. (2002) Embo J 21, 4037-4048). The effect of Akt on AR ubiquitylation and degradation seems to be dependent on AR phosphorylation, since activation of Akt did not induce ubiquitylation or degradation of mtAR, which lacks Akt mediated phosphorylation. Interestingly, the AR mutant was remarkably stable compared with wtAR, indicating that phosphorylation of AR by Akt reduces AR stability (Lin, H. K., et al. (2002) Embo J 21, 4037-4048).

230. Mdm2, a Ring Finger protein, consists of an E3 ligase and suppresses p53 activity by regulation of ubiquitylation and degradation of p53 (Honda, R., et al. (1997) FEBS Lett 420, 25-27; Fang, S., et al. (2000) J Biol Chem 275, 8945-8951). In addition to regulation of p53 function, Mdm2 can also regulate AR activity via regulation of ubiquitylation and degradation of the AR (Lin, H. K., et al. (2002) Embo J 21, 4037-4048). Mdm2 was identified as an E3 ligase for AR and a mediator for Akt-induced AR ubiquitylation and degradation (Lin, H. K., et al. (2002) Embo J 21, 4037-4048). AR protein normally undergoes degradation several hours after its synthesis in cells. However, the signals responsible for AR turnover remain unclear. Based on the data presented herein, the PI3K/Akt/Mdm2 pathway represents an important mechanism to control AR turnover rate. When LNCaP cells are cultured in normal medium, growth factors, such as IGF-1, can activate the PI3K/Akt pathway, which can then be responsible for the turnover of AR protein. In support of this, blockage of the PBK/Akt pathway by LY294002 in LNCaP cells leads to increased AR protein levels (Lin, H. K., et al. (2002) Embo J 21, 4037-4048).

231. Since the PI3K/Akt pathway differentially regulates AR activity in different passage numbers of LNCaP cells (FIGS. 8A-D), it was next determined whether the PI3K/Akt pathway has a distinct effect on AR degradation in these cells. cAkt downregulated AR protein levels in low passage LNCaP, but slightly enhanced AR protein levels in high passage LNCaP cells (FIG. 10A). In contrast, LY294002 enhanced AR protein levels in low passage LNCaP cells, but slightly reduced AR protein levels in high passage LNCaP cells (FIG. 10B). To prove the role of Akt in regulation of AR degradation directly, the effect of Akt on AR protein stability was examined. Overexpression of cAkt in low passage LNCaP cells led to accelerated AR degradation (FIG. 10C, left panel). cAkt did not promote AR degradation in high passage LNCaP cells, but slightly stabilized AR stability (FIG. 10C, right panel), which indeed correlated with the effect of PI3K/Akt on AR transcriptional activity in FIGS. 8A-D and AR protein levels in FIGS. 10A-B. These results indicate that the PI3K/Akt pathway induces AR degradation in low passage LNCaP cells, but not in the high passage LNCaP cells.

### c) Summary

232. Based on this study and previous reports (Lin, H. K., et al. (2001) Proc Natl Acad Sci USA 98, 7200-7205; Lin, H. K., et al. (2002) Embo J 21, 4037-4048) a model for the PI3K/Akt pathway action on regulation of AR activity in prostate cancer LNCaP cells (FIG. 11) is proposed. The PI3K/Akt pathway exhibits a cell passage-dependent regulation of AR activity. In low passage LNCaP cells, the basal activity of PI3K/Akt signaling is low and cells are strongly dependent on androgen signaling for growth and survival. However, in high passage LNCaP cells, the basal activity of the PI3K/Akt pathway is high and cells are less dependent on androgen signaling. The PI3K/Akt pathway not only provides the growth and survival signals for prostate cancer cells, but also enhances AR activity in high passage LNCaP cells via an unknown mechanism.

233. The PI3K/Akt pathway provides a survival and growth signal for prostate cancer cells and induces AR activation in the presence or absence of androgen. Given its activation during prostate cancer progression, PB3K/Akt signaling represents a new chemotherapeutic target with the potential to be particularly effective. It may be able to combine the therapy that suppresses the PI3K/Akt pathway with the classic androgen ablation therapy to reach the maximal effect in the battle of prostate cancer.

### G. Sequences

The following sequences and their accession numbers are representative of the molecules disclosed herein that are a part of the MAP kinase pathway and androgen receptor pathways discussed herein.
SEQ ID NO. 1 MAP kinase kinase nucleotide sequence (Accession No. X96757)
SEQ ID NO. 2 MAP kinase kinase amino acid sequence
SEQ ID NO. 3 MAP kinase-interacting serine/threonine kinase 2 nucleotide sequence (Accession No. BC010256)
SEQ ID NO. 4 MAP kinase-interacting serine/threonine kinase 2 amino acid sequence
SEQ ID NO. 5 MAP-kinase activating death domain nucleotide sequence (Accession No. BC003255)
SEQ ID NO. 6 MAP-kinase activating death domain amino acid sequence
SEQ ID NO. 7 mitogen-activated protein kinase kinase kinase 1(MAP3K1) nucleotide sequence (Accession No. XM_{—}042066)
SEQ ID NO. 8 mitogen-activated protein kinase kinase kinase 1(MAP3K1) amino acid sequence
SEQ ID NO. 9 DKFZp762P223 nucleotide sequence (Accession No. AL834303)
SEQ ID NO. 10 DKFZp762P223 amino acid sequence
SEQ ID NO. 11 mitogen-activated protein kinase kinase kinase 2 (MAP3K2) nucleotide sequence (Accession No. NM_{—}006609)
SEQ ID NO. 12 mitogen-activated protein kinase kinase kinase 2 (MAP3K2) amino acid sequence
SEQ ID NO. 13 Soares_Dieckgraefe_colon_NHCD Homo sapiens cDNA clone nucleotide sequence(Accession No. AI672915)
SEQ ID NO. 14 MEK kinase 1 (MEKK1) nucleotide sequence (Accession No. AF042838)
SEQ ID NO. 15 MEK kinase 1 (MEKK1) amino sequence
SEQ ID NO. 16 qn57e12.x1 NCI_CGAP_Kid5 nucleotide sequence(Accession No. AI302081)
SEQ ID NO. 17 MEK kinase 3 nucleotide sequence (Accession No. U78876)
SEQ ID NO. 18 MEK kinase 3 amino acid sequence
SEQ ID NO. 19 ERK activator kinase (MEK2) nucleotide sequence (Accession No. L11285)
SEQ ID NO. 20 ERK activator kinase (MEKI) nucleotide sequence (Accession No. L11284)
SEQ ID NO. 21 MDM2 nucleotide sequence (Accession No.
SEQ ID NO. 22 MDM2 amino acid sequence
SEQ ID NO. 23 Human protein-serine/threonine (AKT2) nucleotide sequence (Accession No. M95936)
SEQ ID NO. 24 serine/threonine (AKT2) amino acid sequence
SEQ ID NO. 25 serine/threonine kinase Akt-3 nucleotide sequence (Accession No. AJ245709)
SEQ ID NO. 26 serine/threonine kinase Akt-3 amino acid sequence
SEQ ID NO. 27 serine/threonine protein kinase (akt1)nucleotide sequence (Accession No. AF039943)
SEQ ID NO. 28 serine/threonine protein kinase (akt1) amino acid sequence

What is claimed is:

1. A composition comprising a kinase pathway inhibitor and an anti-prostate cancer compound.

2. The composition of claim 1, wherein the anti-prostate cancer compound is an antiandrogen.

3. The composition of claim 2, wherein the anti-androgen is Flutamide, Casodex, or Nilutamide.

4. The composition of claim 2, wherein the anti-androgen is Flutamide.

5. The composition of claim 2, wherein the concentration of the anti-androgen is less than or equal to 20 μM.

6. The composition of claim 1, wherein the kinase pathway inhibitor comprises a MAP kinase inhibitor.

7. The composition of claim 6, wherein the MAP kinase pathway inhibitor comprises U0126.

8. The composition of claim 6, wherein the concentration of the MAP kinase pathway inhibitor is less than or equal to 100 μM.

9. The composition of claim 1, wherein the kinase pathway inhibitor comprises a phosphatidylinositol 3-kinase (PI3K)/Akt inhibitor.

10. The composition of claim 9, wherein the PI3K/Akt inhibitor is selected from the group consisting of SH-5, SH-6; 1L-6-hydroxymethyl-chiro-inositol 2(R)-2-O-methyl-3-O-octadecylcarbonate, SRI 3668, wortmannin, LY294002, and API-59.

11. The composition of claim 10, wherein the PI3K/Akt inhibitor is LY294002.

12. The composition of claim 9, wherein the concentration of the PI3k/Akt kinase pathway inhibitor is less than or equal to 20 μM

13. The composition of claim 1, wherein the anti-prostate cancer compound is less than or equal to 20 μM.

14. The composition of claims 1-13, wherein the composition further comprises a pharmaceutically acceptable carrier.

15. A method of treating a subject with prostate cancer comprising administering the composition of claims 1-13.

16. The method of claim 15 wherein administering the composition comprises injecting the composition into the subject.

17. The method of claim 15, wherein administering the composition comprises taking the composition orally, taking by skin patch, or taking by subcutaneous injection.

18. A method of identifying an inhibitor of the MAP kinase pathway, comprising incubating a library of molecules with a cell comprising an activatable MAP kinase pathway and wherein the cell is also incubated with an antiandrogen, and selecting those molecules which inhibit the activation of the MAP kinase pathway.

19. A method of identifying a prostate cancer inhibitor comprising incubating a cell with hydroxyflutamide, incubating the cell with a potential inhibitor, and assaying the level of activation of a MAP kinase pathway.

20. The method of claim 19, wherein the cell is a DU145 cell.

21. A method of identifying a prostate cancer inhibitor comprising incubating a cell with hydroxyflutamide, incubating the cell with a potential inhibitor, and assaying the level of activation of a PI3K/Akt kinase pathway.

22. The method of claim 21, wherein the cell is a high passage LNCaP cell.

23. A method of reducing the number of prostate cancer cells in a sample comprising contacting the cells with the composition of claim 14.

24. A method of treating a patient with prostate cancer comprising administering the composition of claim 14.


SEQUENCE LISTING (28 sequences)

SEQ ID NO: 1 - DNA, 1587 bp, Artificial Sequence
  Description of Artificial Sequence/note = synthetic construct
cttttagctgccagccctggcccatcatgtagctgcagcacagccttccctaacgttgcaactgggggaaaaatcactttccagtctgttttgcaaggtgtgcatttccatcttgattccctgaaagtccatctgctgcatcggtcaagagaaactccacttgcatgaagattgcacgcctgcagcttgcatctttgttgcaaaactagctacagaagagaagcaaggcaaagtcttttgtgctcccctcccccatcaaaggaaaggggaaaatgtctcagtcgaaaggcaagaagcgaaaccctggccttaaaattccaaaagaagcatttgaacaacctcagaccagttccacaccacctagagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaaaaagcagtggacttaatcggttgaccctactgtggattggtgggtttcggggtgaagcaagttcactacagcatcaatagaaagtcatctttgagataatttaaccctgcctctcagagggttttctctcccaattttctttttactccccctcttaagggggccttggaatctatagtatagaatgaactgtctagatggatgaattatgataaaggcttaggacttcaaaaggtgattaaatatttaatgatgtgtcatatgaaaaaaaaaaaaaaaaaaaaaaaaaaaaaaaaaaaaaaaaaaaaaa

SEQ ID NO: 2 - protein, 334 aa, Artificial Sequence
  Description of Artificial Sequence/note = synthetic construct
MSQSKGKKRNPGLKIPKEAFEQPQTSSTPPRDLDSKACISIGNQNFEVKADDLEPIMELGRGAYGVVEKMRHVPSGQIMAVKRIRATVNSQEQKRLLMDLDISMRTVDCPFTVTFYGALFREGDVWICMELMDTSLDKFYKQVIDKGQTIPEDILGKIAVSIVKALEHLHSKLSVIHRDVKPSNVLINALGQVKMCDFGISGYLVDSVAKTIDAGCKPYMAPERINPELNQKGYSVKSDIWSLGITMIELAILRFPYDSWGTPFQQLKQVVEEPSPQLPADKFSAEFVDFTSQCLKKNSKERPTYPELMQHPFFTLHESKGTDVASFVKLILGD

SEQ ID NO: 3 - DNA, 3080 bp, Artificial Sequence
  Description of Artificial Sequence/note = synthetic construct
gcccagcacagggactctgacttcagcccacagtgtgaagcccgacctgacatgccttccagccagcccattgacatcccagatgccaagaagagaggccggaaaaagaagcgctgtcgggctactgacagcttctcaggcaggttcgaagatgtctatcagctgcaggaggatgtgctgggggaaggtgctcacgctcgtgtgcagacctgtgtcaatctcatcaccaaccaggaatatgctgtcaagatcattgagaagcagctgggccacatccgcagcagggtgttccgggaggtggagatgctgtaccagtgccagggacataggaatgttctagaactgattgagttctttgaggaggaggaccgtttctacctggtgtttgagaagatgcgtggcggatccatcctaagccacatccatagaaggcgccactttaacgagctggaggccagcgtggtagtacaggacgtggccagtgccctggacttcctgcataacaaaggcatcgcccacagggacctaaagccagagaacatcctatgtgagcaccccaaccaggtctcgccagtgaagatctgcgacttcgaccttggcagtggtatcaaactcaatggagactgctcccccatctccacaccagagctgctcaccccgtgtgggtcagctgagtacatggccccagaggtggtggaggccttcagtgaagaggccagcatctacgacaagcgctgcgacctgtggagcctgggcgtcatcctctacatcctgcttagtggctacccgcccttcgtgggccactgtggcagcgactgtggctgggaccgtggggaggcctgtcctgcctgccagaacatgctgtttgagagcatccaggagggcaagtatgagttccctgacaaggactggtcccacatctcctttgctgccaaagacctcatctccaagctgctggtccgagatgccaagcagaggctgagtgctgcccaagtcctgcagcatccctgggtgcaggggtgtgccccagagaacaccctaccgacacccttggttctgcagaggaacagctgtgccaaagacctcacgtcctttgcggctgaggccatcgccatgaaccggcagctggcccagtgtgaggaggacgctgggcaggaccagcctgtggtcatccgagctacctcacgctgcctgcagctgtccccaccctcccagtccaagctggcccagcggcgccagagggctagcctgtcggccacccctgtggtccttgtgggggatcgcgcatgacccccactagctccttgtacatatgcccctgccccgcggggcctgaaggctagggacctggacaccccaccccttgccattccaggtgccagctcagctgggtcctctgggggtgtaggggtctgttagggggtgtctccttttctccctgtccttcccctgccctgcccacttggctttgttttgtttgtttttctttgccgctattgaaagcaagtgcccggaggagggcggagggctcaggccgcccagcctgcaccccacgatgctcacctgccaactgtgaaggtcctgccacctgcgcccccacctccactccagccactctgctgtcttccagggttggggatcccgcagggtcagcaccccacacctctcccagccctcagtgttgtcagggacaggccctcctggtgagcacagtggtggttgcatctcctcaccagagcacccttgggtctggggtagggcagggctccctgtcttggatagagacctctggggagcaggtggatggggacagtgcacttgattgaccccgagtccccatcatccacctgcagtgtcccttggagggttgacaatcagaaacccctcccaggctgcttagctccttgccctgggacagacctactgctcccaaccccacttcccaggggcagagctggaaggggaccctgcacccagctagctccaccacagcaggagaggtgctggaccaggctttcatcagcaaacatggggctcccacatgtctccccacccagggcacctgagtgccccttctcagggctcagcctgaccacggccacgtcctgcccctggggttcctaagctctcctagctgcttctgttagccagagctgaggccatacccagggctctcaccttcctgttgcccccagagggcagcagctcaggcgtgcctgctttcaggaaagggaggctgggaagggatgtggtggccctgcggtgcccagacctaactgcccgaggcctgtagactgttctagccgaactactatgcaatacaagttcccattttctccatggccctgctggtcgggggcggctgccaggggccaggccaccctgccctgcaactgctcaggtgtctacagggcagcccctggcctcaaaaatccttggtcaggattgtttgtcgagtttagtttaggctttttttttttttttaaagaaataatttgacttgcttccctgttcttgaagagtacttgaatgtcggggtctggtgggtgggggcctgggacacccactgcccagcatcctccaccctcctccctagtctcataggatcgtcacagtggaggtgacatgccttctccagtcctgccccacctgcctctgtggacacatttccaaagaacccctgggggtgggacctcctccatcagtatgactcagctgttggccacctgaggactcggcccccctgcaggttcctgaagcaacctgactgggcagtgagcagcattgacccccactcacccccaaaacagggctgtgatttccttagtccttccaagcccgacctggaggatgggtcagaccccttaactgtgaatgagacatgatcctgggctggcttcgccacaaaccatgcagaaatctaaaaggcctgttgtagagtgggggacatgcaagcacttttaactccatcgtaccaggtgaactgacctccggactcctttcccaccaactgtcaacgccaggattttgtattctgttttgtaaggatttaataaaagtcatttaaaaaaaaaaaaaaa

SEQ ID NO: 4 - protein, 412 aa, Artificial Sequence
  Description of Artificial Sequence/note = synthetic construct
MPSSQPIDIPDAKKRGRKKKRCRATDSFSGRFEDVYQLQEDVLGEGAHARVQTCVNLITNQEYAVKIIEKQLGHIRSRVFREVEMLYQCQGHRNVLELIEFFEEEDRFYLVFEKMRGGSILSHIHRRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGHCGSDCGWDRGEACPACQNMLFESIQEGKYEFPDKDWSHISFAAKDLISKLLVRDAKQRLSAAQVLQHPWVQGCAPENTLPTPLVLQRNSCAKDLTSFAAEAIAMNRQLAQCEEDAGQDQPVVIRATSRCLQLSPPSQSKLAQRRQRASLSATPVVLVGDRA

SEQ ID NO: 5 - DNA, 1096 bp, Artificial Sequence
  Description of Artificial Sequence/note = synthetic construct
ccagactcagaggagaggggcgaatcacaagcgacagctcctgttcactgggccagcggcttaggtgctccttccctttaatgatccagattatatcagaattcctcctgggaatgctggcttcttgctcagtgccctgaagtttctctgcaatgaactgacaaatcggaaccatggtgcaaaagaagttctgccctcggttacttgactatctcgtgatcgtaggggcgaggcacccaagcagtgacagtgtggctcagactcctgagctgctgcggaggtacccactagaggatcacccagagttccccctgcccccagatgtggtgttcttctgccagccagaaggatgtctgagtgtgcggcagcggcggatgagccttcgggacgatacctcttttgtcttcaccctaaccgataaggacaccggagtcacccgctatggcatctgtgtcaacttctaccgttccttccaaaagcgaatgccaaaggaaaaggtggaaggcggagcaggaccccgtgggaaggaaggcgctcacacctctggtgcctcagaagaggctgccgctgggagctcagagagtggctcaaccttgcagccgcctagtgctgactccactcctgacgtaaaccagtctcctcggggcaaacgtagggcaaaagcgggcagccgctcccgcaacagtaccctgacatccctgtgtgtgcttagccactaccctttcttctctaccttcagagagtgtctgtatactctcaaacgtttggtagactgctgtagtgaacggctgctaggcaagaaactgggcatccctcgaggtgtacaaaggtacggtttgctgcttgtgcttgggagaacattaagagattgatattacttcgtgagaggaaaagaaatttcctctagggctggagaggtggtaagagcactgaccgctcttccagaggtccccagttcaattcccagcaattacatggtggctcacaacattctgtaatggggtcggatgccctcttctggtgtgtctgaagacagtgatagtatatataaaataactcacagatataaaataaatctttaaaaaaaaaaaaaaaa

SEQ ID NO: 6 - protein, 232 aa, Artificial Sequence
  Description of Artificial Sequence/note = synthetic construct
MVQKKFCPRLLDYLVIVGARHPSSDSVAQTPELLRRYPLEDHPEFPLPPDVVFFCQPEGCLSVRQRRMSLRDDTSFVFTLTDKDTGVTRYGICVNFYRSFQKRMPKEKVEGGAGPRGKEGAHTSGASEEAAAGSSESGSTLQPPSADSTPDVNQSPRGKRRAKAGSRSRNSTLTSLCVLSHYPFFSTFRECLYTLKRLVDCCSERLLGKKLGIPRGVQRYGLLLVLGRTLRD

SEQ ID NO: 7 - DNA, 7161 bp, Artificial Sequence
  Description of Artificial Sequence/note = synthetic construct
atgggcagccaggccctgcaggagtggggacagagggaacccggccggtggcccgaccctgcagggaagaaggacgtgcggcgagaagcatcggattcggggagggccgggacctggccgagggggccgagcgaatgtagcccgcgagagaaaatggcggcggcggcggggaatcgcgcctcgtcgtcgggattcccgggcgccagggctacgagccctgaggcaggcggcggcggaggagccctcaaggcgagcagcgcgcccgcggctgccgcgggactgctgcgggaggcgggcagcgggggccgcgagcgggcggactggcggcggcggcagctgcgcaaagtgcggagtgtggagctggaccagctgcctgagcagccgctcttccttgccgcctcaccgccggcctcctcgacttccccgtcgccggagcccgcggacgcagcggggagtgggaccggcttccagcctgtggcggtgccgccgccccacggagccgcgagccgcggcggcgcccaccttaccgagtcggtggcggcgccggacagcggcgcctcgagtcccgcagcggccgagcccggggagaagcgggcgcccgccgccgagccgtctcctgcagcggcccccgccggtcgtgagatggagaataaagaaactctcaaagggttgcacaagatggatgatcgtccagaggaacgaatgatcagggagaaactgaaggcaacctgtatgccagcctggaagcacgaatggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacgtggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacgtctagttcagaaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattttctagaaactggaatgtgagagagatggccctcaggcgtctttcccatgatgtcagtggggccctgctgttggcaaatggggagagcactggaaattctgggggcagcagtggaagcagcccgagtgggggagccaccagtgggtcttcccagaccagtatctcaggagatgtggtggaggcatgctgcagcgttctgtcaatggtctgtgctgaccctgtctacaaagtgtacgttgctgctttaaaaacattgagagccatgctggtatatactccttgccacagtttagcggaaagaatcaaacttcagagacttctccagccagttgtagacaccatcctagtcaaatgtgcagatgccaatagccgcacaagtcagctgtccatatcaacactgttggaactgtgcaaaggccaagcaggagagttggcagttggcagagaaatactaaaagctggatccattggtattggtggtgttgattatgtcttaaattgtattcttggaaaccaaactgaatcaaacaattggcaagaacttcttggccgcctttgtcttatagatagactgttgttggaatttcctgctgaattttatcctcatattgtcagtactgatgtttcacaagctgagcctgttgaaatcaggtataagaagctgctgtccctcttaacctttgctttgcagtccattgataattcccactcaatggttggcaaactttccagaaggatctacttgagttctgcaagaatggttactacagtaccccatgtgttttcaaaactgttagaaatgctgagtgtttccagttccactcacttcaccaggatgcgtcgccgtttgatggctattgcagatgaggtggaaattgccgaagccatccagttgggcgtagaagacactttggatggtcaacaggacagcttcttgcaggcatctgttcccaacaactatctggaaaccacagagaacagttcccctgagtgcacagtccatttagagaaaactggaaaaggattatgtgctacaaaattgagtgccagttcagaggacatttctgagagactggccagcatttcagtaggaccttctagttcaacaacaacaacaacaacaacaacagagcaaccaaagccaatggttcaaacaaaaggcagaccccacagtcagtgtttgaactcctctcctttatctcatcattcccaattaatgtttccagccttgtcaaccccttcttcttctaccccatctgtaccagctggcactgcaacagatgtctctaagcatagacttcagggattcattccctgcagaataccttctgcatctcctcaaacacagcgcaagttttctctacaattccacagaaactgtcctgaaaacaaagactcagataaactttccccagtctttactcagtcaagacccttgccctccagtaacatacacaggccaaagccatctagacctaccccaggtaatacaagtaaacagggagatccctcaaaaaatagcatgacacttgatctgaacagtagttccaaatgtgatgacagctttggctgtagcagcaatagtagtaatgctgttatacccagtgacgagacagtgttcaccccagtagaggagaaatgcagattagatgtcaatacagagctcaactccagtattgaggaccttcttgaagcatctatgccttcaagtgatacaacagtaacttttaagtcagaagttgctgtcctgtctcctgaaaaggctgaaaatgatgatacctacaaagatgatgtgaatcataatcaaaagtgcaaagagaagatggaagctgaagaagaagaagctttagcaattgccatggcaatgtcagcgtctcaggatgccctccccatagttcctcagctgcaggttgaaaatggagaagatatcatcattattcaacaggatacaccagagactctaccaggacataccaaagcaaaacaaccgtatagagaagacactgaatggctgaaaggtcaacagataggccttggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggcctttcgtatctccatgaaaaccaaatcattcacagagatgtcaaaggtgccaatttgctaattgacagcactggtcagagactaagaattgcagattttggagctgcagccaggttggcatcaaaaggaactggtgcaggagagtttcagggacaattactggggacaattgcatttatggcacctgaggtactaagaggtcaacagtatggaaggagctgtgatgtatggagtgttggctgtgctattatagaaatggcttgtgcaaaaccaccatggaatgcagaaaaacactccaatcatcttgctttgatatttaagattgctagtgcaactactgctccatcgatcccttcacatttgtctcctggtttacgagatgtggctcttcgttgtttagaacttcaacctcaggacagacctccatcaagagagctactgaagcatccagtctttcgtactacatggtagccaattatgcagatcaactacagtagaaacaggatgctcaacaagagaaaaaaaacttgtggggaaccacattgatattctactggccatgatgccactgaacagctatgaacgaggccagtggggaacccttacctaagtatgtgattgacaaatcatgatctgtacctaagctcagtatgcaaaagcccaaactagtgcagaaactgtaaactgtgcctttcaaagaactggccctaggtgaacaggaaaacaatgaagtttgcatgactaaattgcagaagcataattttatttttttggagcactttttcagcaatattagcggctgaggggctcaggatctattttaatatttcaattattcttccatttcatatagtgatcacaagcagggggttctgcaattccgttcaaattttttgtcactggctataaaatcagtatctgcctcttttaggtcagagtatgctatgagtagcaatacatacatatatttttaaaagttgatacttctttatgacccacagttgacctttattttcttaaataccagggcagttgtggctcattgtgcattttactgttggcccattcatttcgtttttggaaattatggttttgtattttcatgtttatttacattcatttttgtttattcagggaaagctgatctttttttcaaaccagaaaaaaaaaatgaactagatatgaagtagagttcattaaatatcttgctattgtcagagtttttaaaatatagacttaattttgtttttttaaattggaatacaataaagtactacctacatttgagtcagtcaccactcttattgtgcaggttaagtacaagttaactaaaaataaactgtcctctctggtgcaactcacaaccaagatcaagattaccttaaaatttatttgaattttttagatgttttggttgtcaaactgtaggaaacttcacaacatttaagtcttactctgtatgtaacaatccatcattcaccttcactactggtagtaacatagagctgccattttccttttaccatgcatcatctctttacagtaggcctggcagatcattttttaaaaagattattcaactaccaatcagtaatgtttttaaacagtacatttgctttgaacttggaaaatgtgttcagaaagaaaaatggaattgaatttcatttatacactaattccttggattttgcacagttacctaacggttttagtctggagttaaattcagatgcatggaatcctgaaggaaaatggtagctttttaatctttttgtgtgtgtgtgagtcttttaaatcaagtactgattaactattaagtacaactttgagattttagttttaactcttcagaagccagtgtgaaatagaattggttattctcaaagactcaggataaactaaataagctatatatagagtacatttaaaatgtacaacacaaattggaaataaaataagttacaagataagtttacagggatatattgcttacaatttttaaaaggcagtttgttttttatgtgaatatgtttcttagtgaaattttacattcctttgttttggaagattggcgatatttgaagagttaaaaatagtacagaaatgtgaagtttggtatctctaaatgtgttgtacttgactttcttttttattttgtttttttttttttttgactacttagaattttcacaattctaataagattgtttccaagtctctcatgtgcaagctttaaaggatgcactcttgccattttatgtactggaagatcattggtcagatgaatactgtgtctgacaaaaatgtaaactgtataaactgaggaacctcagctaatcagtattactttgtagatcaccatgcccaccacatttcaaactcaaactatctgtagatttcaaaatccattgtgtttgagtttgtttgcagttccctcagcttgctggtaattgtggtgttttgttttttgttttgttttcaatgcaaatgtgatgtaatattcttattttctttggatcaaagctggactggaaattgtatcgtgtaattatttttgtgttcttaatgttatttggtactcaagttgtaaataacgtctactactgtttattccagtttctactacctcaggtgtcctatagatttttcttctaccaaagttcactttcacaatgaaattatatttgctgtgtgactatgattcctaagatttccagggcttaagggctaacttctattagcaccttactgtgtaagcaaatgttacaaaaaaaaaaaaaaaaaatctctgggttaagaaaatttggcttaaatgtatcctttgttattttaaatatattgagatattttaattaaaatttttaccccattgaaccgattttatagtatttgtacctattttggtgtttttgtctttatagtaaataaaagtttttgaac

SEQ ID NO: 8 - protein, 1563 aa, Artificial Sequence
  Description of Artificial Sequence/note = synthetic construct
MGSQALQEWGQREPGRWPDPAGKKDVRREASDSGRAGTWPRGPSECSPREKMAAAAGNRASSSGFPGARATSPEAGGGGGALKASSAPAAAAGLLREAGSGGRERADWRRRQLRKVRSVELDQLPEQPLFLAASPPASSTSPSPEPADAAGSGTGFQPVAVPPPHGAASRGGAHLTESVAAPDSGASSPAAAEPGEKRAPAAEPSPAAAPAGREMENKETLKGLHKMDDRPEERMIREKLKATCMPAWKHEWLERRNRRGPVVVKPIPVKGDGSEMNHLAAESPGEVQASAASPASKGRRSPSPGNSPSGRTVKSESPGVRRKRVSPVPFQSGRITPPRRAPSPDGFSPYSPEETNRRVNKVMRARLYLLQQIGPNSFLIGGDSPDNKYRVFIGPQNCSCARGTFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVDSPSSLRAAQQQTVQQQPLAGSRRNQESNFNLTHYGTQQIPPAYKDLAEPWIQVFGMELVGCLFSRNWNVREMALRRLSHDVSGALLLANGESTGNSGGSSGSSPSGGATSGSSQTSISGDVVEACCSVLSMVCADPVYKVYVAALKTLRAMLVYTPCHSLAERIKLQRLLQPVVDTILVKCADANSRTSQLSISTLLELCKGQAGELAVGREILKAGSIGIGGVDYVLNCILGNQTESNNWQELLGRLCLIDRLLLEFPAEFYPHIVSTDVSQAEPVEIRYKKLLSLLTFALQSIDNSHSMVGKLSRRIYLSSARMVTTVPHVFSKLLEMLSVSSSTHFTRMRRRLMAIADEVEIAEAIQLGVEDTLDGQQDSFLQASVPNNYLETTENSSPECTVHLEKTGKGLCATKLSASSEDISERLASISVGPSSSTTTTTTTTEQPKPMVQTKGRPHSQCLNSSPLSHHSQLMFPALSTPSSSTPSVPAGTATDVSKHRLQGFIPCRIPSASPQTQRKFSLQFHRNCPENKDSDKLSPVFTQSRPLPSSNIHRPKPSRPTPGNTSKQGDPSKNSMTLDLNSSSKCDDSFGCSSNSSNAVIPSDETVFTPVEEKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDTEWLKGQQIGLGAFSSCYQAQDVGTGTLMAVKQVTYVRNTSSEQEEVVEALREEIRMMSHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

SEQ ID NO: 9 - DNA, 3621 bp, Artificial Sequence
  Description of Artificial Sequence/note = synthetic construct
agaacggtggccggacggagagactgcgggtctgagggactggcgggcgggcgggccgagcggcgccgccgaggccgggctgggccgagcccaggagcgcccgggatgtagcgggccaccctgccgatgccacagcgcccggccgcgggcggagccggagccggagcctggggaggcggcgggggcccagagcgcagcccgcgccccccgcgcggagccaggcccgctgccgtccccgccgcccgggcccccggcatgcagccccggctgcggaggtgacactcacggaccttagccaccgccgccgccatcgccaccatggacgaacaggaggcattgaactcaatcatgaacgatctggtggccctccagatgaaccgacgtcaccggatgcctggatatgagaccatgaagaacaaagacacaggtcactcaaataggcagaaaaaacacaacagcagcagctcagcccttctgaacagccccacagtaacaacaagctcatgtgcaggggccagtgagaaaaagaaatttttgagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctggggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagcagacagcccatccttccggaaatcacgaatgtcccgtgcccagagcttccctgacaacagacaggaatactcagatcgggaaactcagctttatgacaaaggggtcaaaggtggaacctacccccggcgctaccacgtgtctgtgcaccacaaggactacagtgatggcagaagaacatttccccgaatacggcgtcatcaaggcaacttgttcaccctggtgccctccagccgctccctgagcacaaatggcgagaacatgggtctggctgtgcaatacctggacccccgtgggcgcctgcggagtgcggacagcgagaatgccctctctgtgcaggagaggaatgtgccaaccaagtctcccagtgcccccatcaactggcgccggggaaagctcctgggccagggtgccttcggcagggtctatttgtgctatgacgtggacacgggacgtgaacttgcttccaagcaggtccaatttgatccagacagtcctgagacaagcaaggaggtgagtgctctggagtgcgagatccagttgctaaagaacttgcagcatgagcgcatcgtgcagtactatggctgtctgcgggaccgcgctgagaagaccctgaccatcttcatggagtacatgccagggggctcggtgaaagaccagttgaaggcttacggtgctctgacagagagcgtgacccgaaagtacacgcggcagatcctggagggcatgtcctacctgcacagcaacatgattgttcaccgggacattaagggagccaacatcctccgagactctgctgggaatgtaaagctgggggactttggggccagcaaacgcctgcagacgatctgtatgtcggggacgggcatgcgctccgtcactggcacaccctactggatgagccctgaggtgatcagcggcgagggctatggaaggaaagcagacgtgtggagcctgggctgcactgtggtggagatgctgacagagaaaccaccgtgggcagagtatgaagctatggccgccatcttcaagattgccacccagcccaccaatcctcagctgccctcccacatctctgaacatggccgggacttcctgaggcgcatttttgtggaggctcgccagagaccttcagctgaggagctgctcacacaccactttgcacagctcatgtactgagctctcacggccacacagctgccggtcgccctttgctgcatggcagggggctgctgctgggctcagtgaagttgctgcttctcccaggcaaggctgtggaccatggagtggcagcccagccagcgtcggtctgtgccccttccgccactggggctcagagccggggtggggtggctgcagcctcaggactgggagcccccagcctgtcagatccaggagctccagtgtcctgagctcagcgtggaggggtaggggctgggaacagtgtgcaaggcagccgtgggccccaccctcggggatgtgtcctgacactgcaattggcaccgaagcccagagggtctgggggcacaagactgacgccagggtatgaagagtgttattttcattcaaagtgttattttgtttttccttccaatgtctggagaccaccagggcatctctgggctggatgagctcccacaagcctgagggaaaggccagcactcgctagcagtggcaggcagaggcccaggctgccgtcccctagagtcccaggttggctctgccagtcctgtcctttaccaaagatgaatgaagcaaatgtcatgctgccttattcagggaaggaggagcctgtcctgcctgtggccatgaccctgcctctcccaggcaggggcccgcgatgtggaactgctgccactgaggggggatccagttttgtcaatgcagttgtctctgttttacaagttggagtcactcttatgctgtacccagtttctaaactggagactgtgtgtgccctctgggctctgagtacccctgctttgggcttgggcctaggctgcattgaaaagagctgaaggttgtggcctttgcgctcctggcccagcctttgttccccactggagcagaaggggagatggacgacacggtcggggcatctggcctggccagtgccctgatcccagagagcccgaggaggtgtctcaggctgcctgagtcgtgacctgctaggccagagcccactccatctggtagaagggaaagcccatatgctaccaccagctgtgtccaaaaccgccagctctgttcttcctcagccagcctcgcccatccccttgaggtctcagcccctttcccttgtagctcctcccctggagggggaatggcagcaggggttggggaaacagcatctccaagcagcttagagttggccatatttacctcagcctgggcgctggtcctttcttccggcccctcccctccaaaatgtgcctattgctagagctcctccctctcaacacccagtttccttgggagttgtcattaaaagaaaaaaaaaaaaaaaaa

SEQ ID NO: 10 - protein, 657 aa, Artificial Sequence
  Description of Artificial Sequence/note = synthetic construct
MDEQEALNSIMNDLVALQMNRRHRMPGYETMKNKDTGHSNRQKKHNSSSSALLNSPTVTTSSCAGASEKKKFLSDVRIKFEHNGERRIIAFSRPVKYEDVEHKVTTVFGQPLDLHYMNNELSILLKNQDDLDKAIDILDRSSSMKSLRILLLSQDRNHNSSSPHSGVSRQVRIKASQSAGDINTIYQPPEPRSRHLSVSSQNPGRSSPPPGYVPERQQHIARQGSYTSINSEGEFIPETSEQCMLDPLSSAENSLSGSCQSLDRSADSPSFRKSRMSRAQSFPDNRQEYSDRETQLYDKGVKGGTYPRRYHVSVHHKDYSDGRRTFPRIRRHQGNLFTLVPSSRSLSTNGENMGLAVQYLDPRGRLRSADSENALSVQERNVPTKSPSAPINWRRGKLLGQGAFGRVYLCYDVDTGRELASKQVQFDPDSPETSKEVSALECEIQLLKNLQHERIVQYYGCLRDRAEKTLTIFMEYMPGGSVKDQLKAYGALTESVTRKYTRQILEGMSYLHSNMIVHRDIKGANILRDSAGNVKLGDFGASKRLQTICMSGTGMRSVTGTPYWMSPEVISGEGYGRKADVWSLGCTVVEMLTEKPPWAEYEAMAAIFKIATQPTNPQLPSHISEHGRDFLRRIFVEARQRPSAEELLTHHFAQLMY

SEQ ID NO: 11 - DNA, 3336 bp, Artificial Sequence
  Description of Artificial Sequence/note = synthetic construct
ttttattaatatgctgatctgttttatcttctcacggccataaagaatgctgatgggagaaccattttcctaattttcaaattgttgagctgtttgccataatggatgatcagcaagctttgaactcaatcatgcaagatttggctgtccttcataaggccagtcgaccagcattatccttgcaggaaaccagaaaagcaaaatcttcatcaccaaaaaaacagaatgatgtccgagtcaaatttgaacatagaggagaaaaaagaatccttcagttccccagaccagttaaactggaagatctgagatctaaagctaaaattgcctttggacagtctatggatctacattataccaataacgagttggtaattccattaactactcaagatgacttggacaaagctctggaactgctggatcgtagtattcatatgaagagcctcaagatattacttgtaataaatggaagtacacaggctactaatttagaaccattgccatcactagaagatttggataatacagtatttggagcagagaggaaaaaacggctatctataataggtcctactagtagagatagaagttctcctcccccgggttacattccagatgaattacaccaggttgcccggaatgggtcattcactagtatcaacagtgaaggagagttcattccagagagcatggaacaaatgctggatccattatctttaagcagccctgaaaattctggctcaggaagttgtccatcacttgatagtcctttggatggagagagctatccaaaatcacgaatgcctagggctcagagctacccagataatcatcaggaattttcagactatgataaccctatctttgagaaatttggaaaaggaggaacatatccaagaaggtatcatgtttcatatcatcatcaagaggtaataatgatggtcgtaaaacttttccaagagctagaaggacccaggggaaccagcttacggtctcctgtgagtttcagtcctactgatcattccttaagcactagtagtggaagcagtatctttaccccagagtatgatgatagtcgaataagaagaaggggaagtgacatagacaatcctactttgacagtaatggacatcagcccacccagccgttcacctcgagctccgaccaactggagattgggcaaactgcttggccaaggagcctttggaagggtctacctctgttatgatgttgatacaggaagagaattggctgttaagcaagttcaatttgaccccgatagtcctgagaccagcaaggaagtaaatgcacttgagtgtgaaattcagttgctgaaaaacttgctacatgagcgaattgttcagtattatggctgtttgagggatccccaggaaaaaacactttccatatttatggaatatatgccagggggttcaattaaggaccaattaaaagcatatggcgctcttactgagaatgtgactaggaaatacacccgtcagattctggagggtgtccattatttgcacagtaatatgattgtcctacgagatatcaaaggcgcaaatatcctgcgagattcaacaggcaacgtcaaactaggagattttggggccagcaaacggcttcagaccatctgtctctcagggacaggaatgaagtctgtcacgggcacaccatactggatgagccctgaagtcatcagtggagaaggctatggaagaaaagcagacatctggagtgttgcatgtactgtggtagaaatgctaactgaaaagccgccttgggctgaatttgaagcaatggctgccatctttaaaatcgccactcagccaacaaacccaaagctgccacctcatgtctcagactatactcgagatttcctcaaacggatttttgtagaggccaaactgagaccttcagctgatgaactcttaaggcacatgtttgtgcattatcactagcagccagtaacctctcctgtgcctctacctagctcccatctattcattcaccttctctctgactgcacttttcttttttataaaaaaagagagatgggggagaaaaaagacaagagggaaagtatttctcttgattcttggttaaatttgtttaataataataatatcctaaattttttatatttaatctttttttcccttacaagaacttgaagttttttttttaatttttataatgtactgatgtggttcagagagataaagcactttagtacatagtcactctttttagtacaaacaaatcatttggaatacctaaagattgtagagtcattccctctatcactgacacatcagtgacgatgggaagacatggaaaacaaggagaagaaaatgatgtataatttgtagtttttagtgatagtatttaaaatatatcctcatttgtggggttgagccctaaactttagtttagggtaggtactcaacttaaagaatataggtttcttcttatatctgtattctttagatcctaacctctgtctaccaacctttttgctcagtaggagtcttgatagaagatatgaatctctgagaggtatgtttatttgttaatcctaaccagtataataagcaaatacactataatagatccacgttactggaatctgtaaaccttgagggatagctttctgcttaaaaacacacacacacacacacacacacggaaaacctttattttaaagtcaagttgtgagcaaatagaaataaaagacaaaaggacatcactcttatcaaatgtgtgagcagtagaagagaccacatttacagtcaatagaaataatgaaaaaaaattaggtgtttagtgtattttaaacagttttgttttgttttacttgagggggacgtcccaaaattaaaggaatggagaaataatcaaaatcatgtataccatcttctatttccagctcctgattccccataggtaacatcccttaggagcgaagagttcaattagtaatgtttatgtgttatgtcaggagatgaaacccttgttcttaggatcacagaatactaaagcacctcaaaaaaacaggtatcatgtgaaacagtggttgccaaaagtggagcgaggatgatttcactaggcatttggcaattcttagagacatttccggttgtcacaattggagggatactagtagcatgaattgggtagaggccagggatgttgctaaatagattataatacacaaggaaagcagcctcaaagaattacccctcccaaaatgtcagaagtgctgaggctgagaaaccctgatgtaaagatcagtcccagttataaactgaaaacagctatttacaaagcagt

SEQ ID NO: 12 - protein, 544 aa, Artificial Sequence
  Description of Artificial Sequence/note = synthetic construct
MDDQQALNSIMQDLAVLHKASRPALSLQETRKAKSSSPKKQNDVRVKFEHRGEKRILQFPRPVKLEDLRSKAKIAFGQSMDLHYTNNELVIPLTTQDDLDKALELLDRSIHMKSLKILLVINGSTQATNLEPLPSLEDLDNTVFGAERKKRLSIIGPTSRDRSSPPPGYIPDELHQVARNGSFTSINSEGEFIPESMEQMLDPLSLSSPENSGSGSCPSLDSPLDGESTDHSLSTSSGSSIFTPEYDDSRIRRRGSDIDNPTLTVMDISPPSRSPRAPTNWRLGKLLGQGAFGRVYLCYDVDTGRELAVKQVQFDPDSPETSKEVNALECEIQLLKNLLHERIVQYYGCLRDPQEKTLSIFMEYMPGGSIKDQLKAYGALTENVTRKYTRQILEGVHYLHSNMIVLRDIKGANILRDSTGNVKLGDFGASKRLQTICLSGTGMKSVTGTPYWMSPEVISGEGYGRKADIWSVACTVVEMLTEKPPWAEFEAMAAIFKIATQPTNPKLPPHVSDYTRDFLKRIFVEAKLRPSADELLRHMFVHYH

SEQ ID NO: 13 - DNA, 444 bp, Artificial Sequence
  Description of Artificial Sequence/note = synthetic construct
ttcaaaagtgttctgtttaatacgctttgtctggtagtgcttgggtgcctgtggttggtttctctcactggaaccagtcctgggccccactcgcctggcttcctccagtcgccccaggtcctggggctggtgtgtcagaagctgcctttgtcctctccattcatccatccttgggcctgtctggcctatgatgccctcattcagctctcagggtccagaggtgactggtgtggatcctgctcgctgtgccaagatggccctccagatgcggcataccatccctcccctgatgcgggtgtagatgaggtcatctcgagtggcataggtgagcagagtgtggagggtgaagctatggttcaacagcatttggatggtgcctgaatccacattcagttcctgtagccactgcaccaggccctggtccgttgaagaagcagtggaggc

SEQ ID NO: 14 - DNA, 4693 bp, Artificial Sequence
  Description of Artificial Sequence/note = synthetic construct
ccgagccctgaggcaggcggcggcggaggagccctcaaggcgagcagcgcgcgcgcggctgccgcgggactgctgcgggaggcgggcagcgggggccgcgagcgggcggactggcggcggcggcagctgcgcaaagtgcggagtgtggagctggaccagctgcctgagcagccgctcttccttgccgcctcaccgccggcctcctcgacttccccgtcgccggagcccgcggacgcagcggggagtgggaccggcttccagcctgtggcggtgccgccgccccacggagccgccagccggcgcggcgcccaccttaccgagtcggtggcggcgccggacagcggcgcctcgagtcccgcagcggccgagcccggggagaagcgggcgcccgccgccgagccgtctcctgcagcggcccccgccggtcgtgagatggagaataaagaaactctcaaagggttgcacaagatggatgatcgtccagaggaacgaatgatcagggagaaactgaaggcaacctgtatgccagcctggaagcacgaatggttggaaaggagaaataggcgagggcctgtggtggtaaaaccaatcccagttaaaggagatggatctgaaatgaatcacttagcagctgagtctccaggagaggtccaggcaagtgcggcttcaccagcttccaaaggccgacgcagtccttctcctggcaactccccatcaggtcgcacagtgaaatcagaatctccaggagtaaggagaaaaagagtttccccagtgccttttcagagtggcagaatcacaccaccccgaagagccccttcaccagatggcttctcaccatatagccctgaggaaacaaaccgccgtgttaacaaagtgatgcgggccagactgtacttactgcagcagatagggcctaactctttcctgattggaggagacagcccagacaataaataccgggtgtttattgggcctcagaactgcagctgtgcacatggaacattctgtattcatctgctatttgtgatgctccgggtgtttcaactagaaccttcagacccaatgttatggagaaaaactttaaagaattttgaggttgagagtttgttccagaaatatcacagtaggcgtagctcaaggatcaaagctccatctcgtaacaccatccagaagtttgtttcacgcatgtcaaattctcatacattgtcatcatctagtacttctacatctagttcagaaaacagcataaaggatgaagaggaacagatgtgtcctatttgcttgttgggcatgcttgatgaagaaagtcttacagtgtgtgaagacggctgcaggaacaagctgcaccaccactgcatgtcaatttgggcagaagagtgtagaagaaatagagaacctttaatatgtcccctttgtagatctaagtggagatctcatgatttctacagccacgagttgtcaagtcctgtggattccccttcttccctcagagctgcacagcagcaaaccgtacagcagcagcctttggctggatcacgaaggaatcaagagagcaattttaaccttactcattatggaactcagcaaatccctcctgcttacaaagatttagctgagccatggattcaggtgtttggaatggaactcgttggctgcttattttctagaaactggaatgtgagagagatggccctcaggcgtctttcccatgatgtcagtggggccctgctgttggcaaatggggagagcactggaaattctgggggcagcagtggaagcagcccgagtgggggagccaccagtgggtcttcccagaccagtatctcaggagatgtggtggaggcatgctgcagcgttctgtcaatggtctgtgctgaccctgtctacaaagtgtacgttgctgctttaaaaacattgagagccatgctggtatatactccttgccacagtttagcggaaagaatcaaacttcagagacttctccagccagttgtagacaccatcctagtcaaatgtgcagatgccaatagccgcacaagtcagctgtccatatcaacactgttggaactgtgcaaaggccaagcaggagagttggcagttggcagagaaatactaaaagctggatccattggtattggtggtgttgattatgtcttaaattgtattcttggaaaccaaactgaatcaaacaattggcaagaacttcttggccgcctttgtcttatagatagactgttgttggaatttcctgctgaattttatcctcatattgtcagtactgatgtttcacaagctgagcctgttgaaatcaggtataagaagctgctgtccctcttaacctttgctttgcagtccattgataattcccactcaatggttggcaaactttccagaaggatctacttgagttctgcaagaatggttactacagtaccccatgtgttttcaaaactgttagaaatgctgagtgtttccagtgtttccactcacttcaccaggatgcgtcgccgtttgatggcttatgcagatgaggtggaaattgccgaagccatccagttgggcgtagaagacactttacaacgacaacaacacaacagcttttgcaggcatctgttcccaacaactatctggaaaccacagagaacagttccccttgagtgcacagtccatttagagaaaactggaaaaggattatgtgctacaaaattgagtgccagttcagaggacatttctgagagactggccaggatttcagtaggaccttctagttcaacaacaacaacaacaacaacaacagagcaaccaaagccaatggttcaaacaaaaggcagaccccacagtcagtgtttgaactcctctcctttatctcatcattcccaattaatgtttccagccttgtcaaccccttcttcttctaccccatctgtaccagctggcactgcaacagatgtctctaagcatagacttcagggattcattccctgcagaataccttctgcatctcctcaaacacagcgcaagttttctctacaattccacagaaactgtcctgaaaacaaagactcagataaactttccccagtctttactcagtcaagacccttgccctccagtaacatacacaggccaaagccatctcgacctaccccaggtaatacaagtaaacagggagatccctcaaaaaatagcatgacacttgatctgaacagtagttccaaatgtgatgacagctttggcttgagcagcaatagtagtaattgctgttataccagtgacgagacagtgttcaccccagtagaggagaaatgcagattagatgtcaatacagagctcaactccagtattgaggaccttcttgaagcatctatgccttcaagtgatacaacagtaacttttaagtcagaagttgctgtcctgtctcctgaaaaggctgaaaatgatgatacctacaaagatgatgtgaatcataatcaaaagtgcaaagagaagatggaagctgaagaagaagaagctttagcaattgccatggcaatgtcagcgtctcaggtagccctccccatagttcctcagctgcaggttgaaaatggagaagatatcatcattattcaacaggatacaccagagactctaccaggacataccaaagcaaaacaaccgtatagagaagacactgaatggctgaaaggtcaacagataggccttggagcattttcttcttgttatcaggctcaagatgtgggaactggaactttaatggctgttaaacaggtgacttatgtcagaaacacatcttctgagcaagaagaagtagtagaagcactaagagaagagataagaatgatgagccatctgaatcatccaaacatcattaggatgttgggagccacgtgtgagaagagcaattacaatctcttcattgaatggatggcagggggatcggtggctcatttgctgagtaaatatggagccttcaaagaatcagtagttattaactacactgaacagttactccgtggactttcgtatctccatgaaaaccaaatcattcacagagatgtcaaaggtgccaatttgctaattgacagcactggtcagagactaagaattgcagattttggagctgcagccaggttggcatcaaaaggaactggtgcaggagagtttcagggacaattactggggacaattgcatttatggcacctgaggtactaagaggtcaacagtatggaaggagctgtgatgtatggagtgttggctgtgctattatagaaatggcttgtgcaaaaccaccatggaatgcagaaaaacactccaatcatcttgctttgatatttaagattgctagtgcaactactgctccatcgatcccttcacatttgtctcctggtttacgagatgtggctcttcgttgtttagaacttcaacctcaggacagacctccatcaagagagctactgaagcatccagtctttcgtactacatggtagccaattatacagatcaactacgtagaaacaggatgctcaacaagagaaaaaaaacttgtggggaaccacattgatatctacggccatgatgccactgaacagctatgaacgaggccagtggggaacccttacctaagtatgtgattgacaaatcatgatctgtacctaagctcagtatgcaaaagcccaaactagtgcagaaact

SEQ ID NO: 15 - protein, 1495 aa, Artificial Sequence
  Description of Artificial Sequence/note = synthetic construct
PSPEAGGGGGALKASSARAAAAGLLREAGSGGRERADWRRRQLRKVRSVELDQLPEQPLFLAASPPASSTSPSPEPADAAGSGTGFQPVAVPPPHGAASRRGAHLTESVAAPDSGASSPAAAEPGEKRAPAAEPSPAAAPAGREMENKETLKGLHKMDDRPEERMIREKLKATCMPAWKHEWLERRNRRGPVVVKPIPVKGDGSEMNHLAAESPGEVQASAASPASKGRRSPSPGNSPSGRTVKSESPGVRRKRVSPVPFQSGRITPPRRAPSPDGFSPYSPEETNRRVNKVMRARLYLLQQIGPNSFLIGGDSPDNKYRVFIGPQNCSCAHGTFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVDSPSSLRAAQQQTVQQQPLAGSRRNQESNFNLTHYGTQQIPPAYKDLAEPWIQVFGMELVGCLFSRNWNVREMALRRLSHDVSGALLLANGESTGNSGGSSGSSPSGGATSGSSQTSISGDVVEACCSVLSMVCADPVYKVYVAALKTLRAMLVYTPCHSLAERIKLQRLLQPVVDTILVKCADANSRTSQLSISTLLELCKGQAGELAVGREILKAGSIGIGGVDYVLNCILGNQTESNNWQELLGRLCLIDRLLLEFPAEFYPHIVSTDVSQAEPVEIRYKKLLSLLTFALQSIDNSHSMVGKLSRRIYLSSARMVTTVPHVFSKLLEMLSVSSVSTHFTRMRRRLMAYADEVEIAEAIQLGVEDTLQRQQHNSFCRHLFPTTIWKPQRTVPLECTVHLEKTGKGLCATKLSASSEDISERLARISVGPSSSTTTTTTTTEQPKPMVQTKGRPHSQCLNSSPLSHHSQLMFPALSTPSSSTPSVPAGTATDVSKHRLQGFIPCRIPSASPQTQRKFSLQFHRNCPENKDSDKLSPVFTQSRPLPSSNIHRPKPSRPTPGNTSKQGDPSKNSMTLDLNSSSKCDDSFGLSSNSSNCCYTSDETVFTPVEEKCRLDVNTELNSSIEDLLEASMPSSDTTVTFKSEVAVLSPEKAENDDTYKDDVNHNQKCKEKMEAEEEEALAIAMAMSASQVALPIVPQLQVENGEDIIIIQQDTPETLPGHTKAKQPYREDTEWLKGQQIGLGAFSSCYQAQDVGTGTLMAVKQVTYVRNTSSEQEEVVEALREEIRMMSHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

SEQ ID NO: 16 - DNA, 37 bp, Artificial Sequence
  Description of Artificial Sequence/note = synthetic construct
tggggtcactcttaacgcagtgtaaaaggtaagccct

SEQ ID NO: 17 - DNA, 2348 bp, Artificial Sequence
  Description of Artificial Sequence/note = synthetic construct
ccgccgcccgggcccccggcatgcagccccggctgcggaggtgacactcacggaccttagccaccgccgccgccatcgccaccatggacgaacaggaggcattgaactcaatcatgaacgatctggtggccctccagatgaaccgacgtcaccggatgcctggatatgagaccatgaagaacaaagacacaggtcactcaaataggcagagtgacgtcagaatcaagttcgagcacaacggggagaggcgaattatagcgttcagccggcctgtgaaatatgaagatgtggagcacaaggtgacaacagtatttggacaacctcttgatctacattacatgaacaatgagctctccatcctgctgaaaaaccaagatgatcttgataaagcaattgacattttagatagaagctcaagcatgaaaagccttaggatattgctgttgtcccaggacagaaaccataacagttcctctccccactctgaggtgtccagacaggtgcggatcaaggcttcccagtccgcaggggatataaatactatctaccagccccccgagcccagaagcaggcacctctctgtcagctcccagaaccctggccgaagctcacctccccctggctatgttcctgagcggcagcagcacattgcccggcaggggtcctacaccagcatcaacagtgagggggagttcatcccagagaccagcgagcagtgcatgctggatcccctgagcagtgcagaaaattccttgtctggaagctgccaatccttggacaggtcagcagacagcccatccttccggaaatcacgaatgtcccgtgcccagagcttccctgacaacagacaggaatactcagatcgggaaactcagctttatgacaaaggggtcaaaggtggaacctacccccggcgctaccacgtgtctgtgcaccacaaggactacagtgatggcagaagaacatttccccgaatacggcgtcatcaaggcaacttgttcaccctggtgccctccagccgctccctgagcacaaatggcgagaacatgggtctggctgtgcaatacctggacccccgtgggcgcctgcggagtgcggacagcgagaatgccctctctgtgcaggagaggaatgtgccaaccaagtctcccagtgcccccatcaactggcgccggggaaagctcctgggccagggtgccttcggcagggtctatttgtgctatgacgtggacacgggacgtgaacttgcttccaagcaggtccaatttgatccagacagtcctgagacaagcaaggaggtgagtgctctggagtgcgagatccagttgctaaagaacttgcagcatgagcgcatcgtgcagtactatggctgtctgcgggaccgcgctgagaagaccctgaccatcttcatggagtacatgccagggggctcggtgaaagaccagttgaaggcttacggtgctctgacagagagcgtgacccgaaagtacacgcggcagatcctggagggcatgtcctacctgcacagcaacatgattgttcaccgggacattaagggagccaacatcctccgagactctgctgggaatgtaaagctgggggactttggggccagcaaacgcctgcagacgatctgtatgtcggggacgggcatgcgctccgtcactggcacaccctactggatgagccctgaggtgatcagcggcgagggctatggaaggaaagcagacgtgtggagcctgggctgcactgtggtggagatgctgacagagaaaccaccgtgggcagagtatgaagctatggccgccatcttcaagattgccacccagcccaccaatcctcagctgccctcccacatctctgaacatggccgggacttcctgaggcgcatttttgtggaggctcgccagagaccttcagctgaggagctgctcacacaccactttgcacagctcatgtactgagctctcacggccacacagctgccggtcgccctttgctgcatggcagggggctgctgctgggctcagtgaagttgctgcttctcccaggcaaggctgtggaccatggagtggcagcccagccagcgtcggtctgtgccccttccgccactggggctcagagccggggtggggtggctgcagcctcaggactgggagcccccagcctgtcagatccaggagctccagtgtcctgagctcagcgtggaggggtaggggctgggaacagtgtgcaaggcagccgtgggccccaccctcggggatgtgtcctgacactgcaattggcaccgaagcccagagggtctgggggcacaagactgacgccagggtatgaagagtgttattttc

SEQ ID NO: 18 - protein, 626 aa, Artificial Sequence
  Description of Artificial Sequence/note = synthetic construct
MDEQEALNSIMNDLVALQMNRRHRMPGYETMKNKDTGHSNRQSDVRIKFEHNGERRIIAFSRPVKYEDVEHKVTTVFGQPLDLHYMNNELSILLKNQDDLDKAIDILDRSSSMKSLRILLLSQDRNHNSSSPHSEVSRQVRIKASQSAGDINTIYQPPEPRSRHLSVSSQNPGRSSPPPGYVPERQQHIARQGSYTSINSEGEFIPETSEQCMLDPLSSAENSLSGSCQSLDRSADSPSFRKSRMSRAQSFPDNRQEYSDRETQLYDKGVKGGTYPRRYHVSVHHKDYSDGRRTFPRIRRHQGNLFTLVPSSRSLSTNGENMGLAVQYLDPRGRLRSADSENALSVQERNVPTKSPSAPINWRRGKLLGQGAFGRVYLCYDVDTGRELASKQVQFDPDSPETSKEVSALECEIQLLKNLQHERIVQYYGCLRDRAEKTLTIFMEYMPGGSVKDQLKAYGALTESVTRKYTRQILEGMSYLHSNMIVHRDIKGANILRDSAGNVKLGDFGASKRLQTICMSGTGMRSVTGTPYWMSPEVISGEGYGRKADVWSLGCTVVEMLTEKPPWAEYEAMAAIFKIATQPTNPQLPSHISEHGRDFLRRIFVEARQRPSAEELLTHHFAQLMY

SEQ ID NO: 19 - DNA, 1576 bp, Artificial Sequence
  Description of Artificial Sequence/note = synthetic construct
gaattcgagccgaccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggttggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatagactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacacgtggcgcctccacccaccctcctgcctcaccctgcggagagcaccgtggcggggcgacagcgcatgcaggaacgggggtctcctctcctgccagtcctggccggggtgcctctggggacgggcgacgctgctgtgtgtggtctcagaggctctgcttccttaggttacaaaacaaaacagggagagaaaagcaaaaaaaaaaaaaaaaaa

SEQ ID NO: 20 - DNA, 2222 bp, Artificial Sequence
  Description of Artificial Sequence/note = synthetic construct
attcggcacgagggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaatttttggtgaatgtgggtagtcattcttacaattgcactgctgttcctgctccatgactggctgtctgcctgtattttcggactttgacatttgacatttggtggactttatcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattatttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggagaaagctaaaaaaaaaaaaaaaaaaaaaaaaaaaaaaaaaaaaaaa

SEQ ID NO: 21 - DNA, 2371 bp, Artificial Sequence
  Description of Artificial Sequence/note = synthetic construct
gcaccgcgcgagcttggctgcttctggggcctgtgtggccctgtgtgtcggaaagatggagcaagaagccgagcccgaggggcggccgcgacccctctgaccgagatcctgctgctttcgagccaggagcaccgtccctccccggattagtgcgtacgagcgcccagtgccctggcccggagagtggaatgatccccgaggcccagggcgtcgtgcttccgcagtagtcagtccccgtgaaggaaactggggagtcttgagggacccccgactccaagcgcgaaaaccccggatggtgaggagcaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctagttgacctgtctataagagaattatatatttctaactatataaccctaggaatttagacaacctgaaatttattcacatatatcaaagtgagaaaatgcctcaattcacatagatttcttctctttagtataattgacctactttggtagtggaatagtgaatacttactataatttgacttgaatatgtagctcatcctttacaccaactcctaattttaaataatttctactctgtcttaaatgagaagtacttggttttttttttcttaaatatgtatatgacatttaaatgtaacttattattttttttgagaccgagtcttgctctgttacccaggctggagtgcagtgggtgatcttggctcactgcaagctctgccctccccgggttcgcaccattctcctgcctcagcctcccaattagcttggcctacagtcatctgccaccacacctggctaattttttgtacttttagtagagacagggtttcaccgtgttagccaggatggtctcgatctcctgacctcgtgatccgcccacctcggcctcccaaagtgctgggattacaggcatgagccaccg

SEQ ID NO: 22 - protein, 491 aa, Artificial Sequence
  Description of Artificial Sequence/note = synthetic construct
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 23 - DNA, 1599 bp, Artificial Sequence
  Description of Artificial Sequence/note = synthetic construct
gagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcgaaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttacccctgcc

SEQ ID NO: 24 - protein, 481 aa, Artificial Sequence
  Description of Artificial Sequence/note = synthetic construct
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 25 - DNA, 1547 bp, Artificial Sequence
  Description of Artificial Sequence/note = synthetic construct
gggagtcatcatgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatatgatgaggatggtatggactgcatggacaatgagaggcggccgcatttccctcaattttcctactctgcaagtggacgagaataagtctctttcattctgctacttcactgtcatcttcaatttattactgaaaatgattcctggacatcaccagtcctagctcttacacatagcaggggca

SEQ ID NO: 26 - protein, 479 aa, Artificial Sequence
  Description of Artificial Sequence/note = synthetic construct
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE

SEQ ID NO: 27 - DNA, 2277 bp, Artificial Sequence
  Description of Artificial Sequence/note = synthetic construct
ctctgggggtgctgctgagcgaggcgagaggcagcctcctcctctcggccagaggcaggagcgaggcgagcgcggggaaggcaggccggcagcccgtgtgagccccatgcggcgagcgcctgcgagaaggggaggaggagccggcccgccgcccgccgcccggggatggtgaggaggcggcgctgcgtgagcccagttaggccttcgctcgggcccgccgccagctctcccttcctccgctcgctcgctccccccttccctctccttccctgccgccgccgccgccgccctcccatcacctcctccccgggctcccgcagccataagtagctgagaaggagaaagacaagaaaaagaacatcccttttgtggacccttctgctggagttcaggaatttcaacggtgatcttttgactgatccaccagcctgataaactgatccaccaagagacattcccgccattatgaatgaagtagcgatagtgaaggaaggatggctccacaaacgaggagaatatatcaaaacatggaggccacggtattttcttttaaagaatgatggcacattcattggctacaaggaacgaccgcaagacgttgaccaacgagaatcacctttaaataacttctcagtagctcagtgccagctgatgaagacagaacgacctaaaccaaacacatttatcattagatgcctccagtggaccacagtaattgaaagaacatttcatgtggagactccagaggagcgggaagaatggacaaaagctatccaaactgttgcagacagcctcaagaaacaggaggaagagatgatggattttagatctggttctcctagtgataattcaggtgctgaagaaatggaagtttctatgacaaagccaaaacacaaagtgaccatgaatgaatttgaataccttaagctactgggaaaaggcacttttggaaaggtcattttagttaaagaaaaagcaaccggacggtattatgctatgaaaattctgaagaaggaagttattgtagcaaaggatgaagtagcacacacgctgacagaaaaccgtgttttacagaactcacggcatccattcttaacagctttaaagtattcctttcagacacacgatcgcttgtgttttgttatggagtatgctaacggaggggagttgtttttccatctgtcgagagagcgtgtattttctgaagaccgggcgcgtttttatggggctgagattgtttcagcgctggattacctgcattcagagaagaatgtggtgtacagagatttgaagctggaaaatcttatgctggataaagacgggcacataaaaattacagactttggactatgtaaagaaggcataaaagatggagcaacaatgaagactttctgtggcactccagagtatcttgcaccagaggtgctggaggataatgactatggtcgtgcagtggactggtggggattaggagttgtgatgtatgaaatgatgtgtggccggctccctttctacaatcaggaccatgaaaagctctttgaactcatccttatggaagagattagatttccacgcactttgtcacctgaagcaaaatctctcttgtcaggtttgctgaagaaagatcctaagcaaaggttaggaggcggtcctgatgatgccaaggagattatgcagcacaaattctttgctggcattgtttggcaagatgtatacgggaagaagcttgtacctccatttaagccacaagttacatctgaaacagatacaagatactttgatgaagaatttacagcacagatgattacaatcactcctcctgaccaagatgacagcatggattgtgtagacaatgagagaagacctcattttcctcagttctcctattcagccagtggaaccgcttaatgttttgcagtgttttcccattcagaaacaaaacagactgcattttggggaccttacttcaatggacactagagaactttctatattatctgaattacaaactgtgtttgtattacgatttagatgaatttctaggaagcctcacagattctgtatttaaaacaattctttgatgcatttttgagaaggaaaacaaatccattcttaaagtattacgtcaaggctcttatgctgaacgaccataggtttttaagaatatgcaccaaaactgtttactttagaattaattaaggcattcaatatcagctataggatctaatcattcctgtcaaaagtttaagaccaggaaccttagagtagaaacgaaac

SEQ ID NO: 28 - protein, 480 aa, Artificial Sequence
  Description of Artificial Sequence/note = synthetic construct
MNEVAIVKEGWLHKRGEYIKTWRPRYFLLKNDGTFIGYKERPQDVDQRESPLNNFSVAQCQLMKTERPKPNTFIIRCLQWTTVIERTFHVETPEEREEWTKAIQTVADSLKKQEEEMMDFRSGSPSDNSGAEEMEVSMTKPKHKVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLSPEAKSLLSGLLKKDPKQRLGGGPDDAKEIMQHKFFAGIVWQDVYGKKLVPPFKPQVTSETDTRYFDEEFTAQMITITPPDQDDSMDCVDNERRPHFPQFSYSASGTA